(19) United States
(12) Patent Application Publication
(10) Pub. No.: US 2020/0260962 A1
(43) Pub. Date: Aug. 20, 2020

(54) SYSTEM AND METHODS FOR ACQUISITION AND ANALYSIS OF HEALTH DATA

(71) Applicant: MAGNIWARE LTD., Toronto (CA)

(72) Inventors: Anthony MOUCHANTAF, Toronto (CA); Miles James MONTGOMERY, Toronto (CA); Alexander Ibrahim MOSA, Toronto (CA); Firas Kamal EDDINE, Toronto (CA); Aniruddha BORAH, Toronto (CA); Wenzhong ZHANG, Toronto (CA)

(21) Appl. No.: 15/774,888

(22) PCT Filed: Nov. 8, 2016
(86) PCT No.: PCT/CA2016/051297

§ 371 Date: May 9, 2018

Related U.S. Application Data
(60) Provisional application No. 62/368,932, filed on Jul. 29, 2016.
(60) Provisional application No. 62/348,599, filed on Jun. 10, 2016.
(60) Provisional application No. 62/286,797, filed on Jan. 25, 2016.
(60) Provisional application No. 62/252,883, filed on Nov. 9, 2015.

(51) Int. Cl. A61B 5/0205; G16H 40/63; A61B 5/00; A61B 5/113

(52) U.S. Cl. CPC A61B 5/0205; G16H 40/63; A61B 5/0004; A61B 5/113; A61B 2562/0219; A61B 5/7275; A61B 5/4809; A61B 5/683; A61B 5/6804; A61B 5/6823

(57) ABSTRACT

An apparatus for measuring cardiopulmonary data of a wearer, comprising: a sensor operable to produce a data stream indicative of movements of a wearer's body; a positioning device holding said sensor proximate an anatomical landmark on said wearer's body for conduction of mechanical vibrations from said wearer's body to said sensor; and a processor configured to receive said data stream and produce a rate signal indicative of cardiac or respiratory rate data of said wearer using an algorithm comprising peak detection;

### RELATED APPLICATIONS

This claims priority from U.S. provisional patent application No. 62/252,883, filed Nov. 9, 2015; U.S. provisional patent application No. 62/286,797, filed Jan. 25, 2016; U.S. provisional patent application No. 62/348,599, filed Jun. 10, 2016; and U.S. provisional patent application No. 62/368,932, filed Jul. 29, 2016, the entire contents of which are incorporated herein by reference.

### FIELD

This relates to acquisition and analysis of health data, in particular, physiological, behavioural and genetic data.

### BACKGROUND

Physical and psychological health is impacted by numerous determinants. Such determinants can include for example, an individual's habits and behaviours and environmental factors. These factors are believed to interact with an individual's genetic makeup and predispositions.

Unfortunately, it has historically been difficult to obtain reliable and detailed genetic, physiological and behavioural data for particular individuals and for populations of individuals. Modeling of interaction between such factors is therefore limited and the interaction is not completely understood.

### SUMMARY

An example apparatus for measuring cardiopulmonary data of a wearer comprises: a sensor operable to produce a data stream indicative of movements of a wearer's body; a positioning device holding said sensor proximate an anatomical landmark on said wearer's body for conduction of mechanical vibrations from said wearer's body to said sensor; and a processor configured to receive said data stream and produce a rate signal indicative of cardiac or respiratory rate data of said wearer using an algorithm comprising peak detection.

An example apparatus for measuring cardiopulmonary data of a wearer comprises: a sensor mounted against a wearer's body to produce a data stream indicative of movements of said wearer's body; a processor configured to receive said data stream and produce a rate signal indicative of cardiac or respiratory rate data of said wearer by correlating segments of said data stream to templates using an algorithm comprising peak detection; a wireless radio for transmitting said rate signal to a computing device.

An example apparatus for measuring cardiopulmonary data of a wearer comprises: a sensor for producing a data stream indicative of cardiac, activity classification, activity level or respiratory data; a processor configured to receive said data stream and produce a rate signal indicative of cardiac or respiratory rate data of said wearer according to one of a first algorithm and a second algorithm less computationally intensive than said first algorithm, wherein said processor is configured to select one of said first and said second algorithms by processing said data stream according to a heuristic relating said data stream to an activity level of a wearer.

An example method of providing health information to a user comprises: receiving a first data set comprising measurements of bodily movements obtained from an accelerometer mounted to the user's body; receiving a second data set comprising genetic data associated with said user; storing said first and second data sets in respective first and second tables in a data store; performing a correlation analysis to identify an association between data of said first table and data of said second table; storing, in said data store, a rule representative of said association; generating a recommendation by comparing said data of said first and second data set to said rule, and transmitting said recommendation to a mobile computing device of said user by way of a communication network.

An example system for acquisition and analysis of health data comprises: a data acquisition device comprising an accelerometer for measuring movements of a user's body, and operable to electronically transmit a data set representing said movements; a data store with a first table for containing said data set and a second table containing genetic data of the user; a processor; a memory containing computer-readable instructions which, when exercised by said processor, cause said processor to: receive said data set by way of said network; perform a correlation analysis to identify an association between data of said first table and data of said second table; store, in said data store, a rule representative of said association; generate a recommendation by comparing said data of said data set and said genetic data of said user to said rule, and transmit said recommendation to a mobile computing device of said user by way of a communication network.

An example apparatus for measuring cardiopulmonary data of a wearer comprises: a sensor operable to produce a data stream indicative of movements of a wearer's body while positioned proximate an anatomical landmark on the wearer's body for conduction of mechanical vibrations from the wearer's body to the sensor; a processor configured to receive the data stream and produce a rate signal indicative of cardiac or respiratory rate data of the wearer using an algorithm comprising peak detection; and a display for presenting feedback based on the data stream.

An example method of measuring cardiopulmonary data of a wearer, comprising: positioning a data acquisition device comprising a sensor proximate an anatomical landmark on the wearer's body for conduction of mechanical vibrations from the wearer's body to the sensor; to produce a data stream indicative of movements of the wearer's body; at a processor, receiving the data stream and producing a rate signal indicative of cardiac or respiratory rate data of the wearer using an algorithm comprising peak detection; and presenting feedback based on the data stream on a display of the data acquisition device.

### BRIEF DESCRIPTION OF DRAWINGS

In the figures, which depict example embodiments:

FIG. 1 is a block schematic diagram of a data acquisition and analysis system;

FIG. 2 is a block diagram of components of a server of the system of FIG. 1;

FIG. 3 is a block diagram of software at the server of FIG. 2;

FIG. 4 is a block diagram of components of a data acquisition device of the system of FIG. 1;

FIG. 5 is a block diagram of software at the data acquisition device of FIG. 4;

FIGS. 6A-6B are schematic views of the data acquisition device of FIG. 4;

FIGS. 7A-7B are side elevation and perspective views of the data acquisition device of FIG. 4;

FIGS. 8A-8B are top elevation and perspective views of a circuit board of the data acquisition device of FIG. 4;

FIGS. 9A-9D are schematic views of holding apparatus illustrative of example anatomical zones/regions of data acquisition;

FIGS. 9E-9G are schematic views of a data acquisition device held by a user;

FIG. 10 is a flowchart that illustrates a process for measuring data of a user;

FIG. 11 is a flowchart illustrating a process for classifying activity of a user;

FIG. 12 is a flowchart illustrating a process for calculating a respiration rate of a user;

FIG. 13 is a flowchart illustrating a process for calculating a heart rate of a user;

FIG. 14 is a flowchart illustrating another process for calculating a heart rate of a user;

FIG. 15 is a flowchart illustrating a process for processing of signals produced by measurement of a user;

FIG. 16 is a flowchart illustrating a process for processing of signals produced by measurement of a user;

FIG. 17 is a trace of an example raw Z Axis Signal from accelerometer 20 second sample;

FIG. 18 is a trace of an acceleration signal Filtered for SCG with band pass of 10 Hz to 35 Hz;

FIG. 19 is a trace of a rectified Signal for envelope detection;

FIG. 20 is a trace of a low pass filtered signal at 2 Hz, and high passed signal at 0.7 Hz;

FIG. 21 is a trace of a filtered SCG signal, post rectification, low pass at 3 Hz, high pass at 0.7 Hz;

FIG. 22 is a trace of a segment of filtered data for template matching;

FIG. 23 is a trace of an extracted best match template from the segment of FIG. 22;

FIG. 24 is a trace of a correlation signal derived using cosine similarity between a match template and a rectified signal segment;

FIG. 25 is a trace of a derived respiration signal after filtering with a low pass filter, and a high pass filter;

FIG. 26 is a trace of a raw Z axis signal, with aliasing noise;

FIGS. 27A-27C are representations of data tables stored at the server of FIG. 2;

FIG. 28 is a flow chart depicting a sample process of sleep scoring;

FIG. 29 is a flow chart depicting a sample process of processing data measured from users; and

FIG. 30 is a flow chart depicting a sample process of providing feedback messages to users.

### DETAILED DESCRIPTION

Embodiments of methods, systems, and apparatus are described through reference to the drawings.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Physiological and behavioural data may include data relating to various types of activities, including, but not limited to, cardiopulmonary activity, bodily movements (e.g., step counts, bodily positioning, changes in orientation, changes in altitude, bodily acceleration, rotation, translation), positional changes, etc. In the context of this disclosure, the term cardiopulmonary may describe aspects related to the cardiovascular system (e.g., blood, heart, blood vessels, heart beats, heart murmurs, irregular blood flow, heart beat variations, heart rate), and/or the pulmonary system (lungs, respiration, trachea, bronchi, bronchioles, diaphragm, alveoli, among others). The term cardiopulmonary means one or both of cardiovascular or pulmonary.

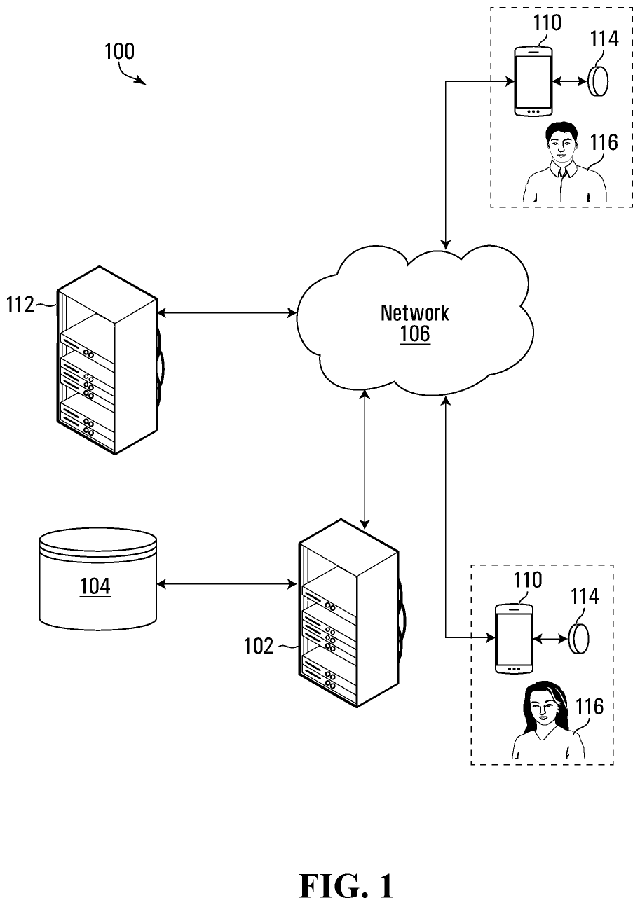

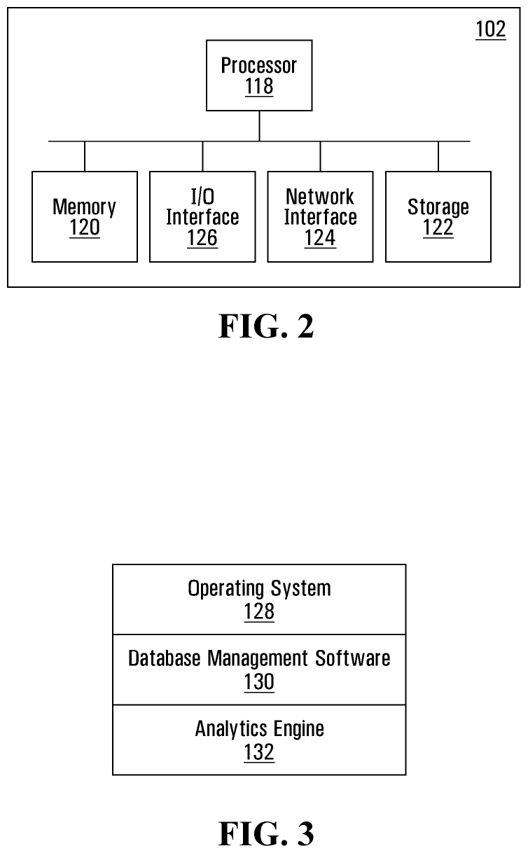

FIG. 1 depicts an example data analysis system 100. Data analysis system 100 includes a server 102, with interconnected data storage 104. Server 102 may be interconnected to a network 106, which may be an IPv4, IPv6, X.25, IPX compliant or similar network, including one or more wired or wireless access points. Network 106 may be a local-area network (LAN) or a wide-area network (WAN), such as the internet, and may be connected with other communications networks, such as GSM/GPRS/3G/4G/LTE networks. Server 102 may host database data in its interconnected data storage 104. As depicted, data analysis system 100 includes a single server 102 with an interconnected data storage 104. However, in other embodiments, more servers 102 and data storage 104 may be present.

Server 102 may communicate with one or more client computing devices 110 and one or more data acquisition facilities 112 over network 106. Client computing devices 110 may be connected directly to network 106, or may be connected to network 106 by way of another network 112, which may be a LAN or a WAN such as the internet. Client computing devices 110 may be, for example personal computers, smartphones, tablet computers, or the like, and may be based on any suitable operating system, such as Microsoft Windows, Apple OS X or iOS, Linux, Android, or the like.

In an example, client computing device 110 may be a smart phone or other mobile computing device. As described in further detail below, client computing device 110 may be associated with a user 116 and may be configured to collect and report to server 102 data related to user 116, such as behavioural, physiological and environmental data.

As depicted, user 116 may have a wearable data acquisition device 114. Data acquisition device 114 is configured to acquire data such as physiological measurements of the user 116. For example, data acquisition device 114 may be configured to measure respiration rate, respiration rate variability, pulse rate, and pulse rate variability. Data acquisition device 114 may be connected in data communication with client computing device 110, for example, using bluetooth or another suitable communication technology. Data acquisition device 114 is configured to acquire and process measurements of user 116, and send acquired and processed data to client computing device 110, which in turn sends the data to server 102 for storage. Alternatively, data acquisition device 114 may communicate directly with server 102 and send data to server 102 over network 106.

Data acquisition device 114 may be utilized, for example, to obtain waveform (e.g., seismocardiography (SCG), among others) readings. Data acquisition device 114 may be configured for application of various algorithms that may aid in the processing, extraction, and/or signal cleaning required to obtain the waveform (e.g., SCG) readings. While SCG is described in the context of waveform readings, some embodiments may not be limited solely to SCG and data acquisition device 114 may be configured to receive, measure and detect other types of waveform readings. Readings may include various types of heart sounds, such as a first heart sound (S1) and a second heart sound (S2), which may be indicative of the beating of a human heart. These may, for example, be produced by the closing of various heart valves, etc. Other sounds and/or artifacts may be present, such as heart murmurs, adventitious sounds, gallop rhythms, etc. The S1 and S2 sounds may occur in pairs.

In the context of SCG, obtaining SCG readings (e.g., acquiring the aortic opening (AO)) may be particularly challenging in view of polymorphism of the SCG signals (e.g., there is significantly more variation in the signal morphology with SCG than with electrocardiogram (ECG) based techniques), various algorithms are therefore disclosed for robustly extracting features from the SCG signals. For example algorithms such as the Pan-Tompkins algorithm may not be robust enough for locating the AO-peaks from the SCG signal. The location of the data acquisition device relative to the user's body may contribute to signal polymorphism. For example, signal polymorphism may differ whether the data acquisition device is located proximate, e.g., the chest, sternum, pectoral region, waist, wrist, etc.

A challenge, for example, may arise from interpersonal variations in the SCG morphology. Morphological variations in the SCG waveform complicate the extraction of reliable vital peak locations and interbeat intervals, which may be especially relevant for people having some heart disease and/or other types of heart conditions. For example, polymorphism may lead to poor performance for beat-beat interval calculation.

Accordingly, currently available devices may require the use of ECG data in conjunction with SCG data. Accurate and reliable heart rate (HR) and heart rate variability (HRV) sensing from SCG may have been difficult to achieve normally requiring concurrent ECG. Relative to obtaining ECG data, obtaining SCG data may be non-invasive and more user friendly to use. For example, obtaining SCG information does not need sensors to be attached to the surface of the skin using electrical conductors.

Further, obtaining ECG data may require specialized monitoring equipment and a number of electrodes attached to the chest, which not only is cumbersome for the wearer. Moreover, body hair may need to be removed in order to make sufficient contact for acquiring a signal.

In some embodiments, devices disclosed herein apply an algorithm to localize the AO-peaks in the SCG signal by using R-peaks located in the ECG signal as fiducial points. The algorithm may be used for beat-to-beat interval estimation in SCG and may be based upon a windowing method proposed. Accordingly, some embodiments of the apparatus 102 may include the recording and processing of SCG data (e.g., using computing devices such as smart phones and/or by means of a specialized (HRV) analysis application and may be used without direct supervision of medical experts. In some embodiments, the data acquisition device 114 may be able to indicate and/or estimate aspects of posture and body position, such as laying down on a back, left, or right side, among other aspects.

Various algorithms may be utilized to aid in the interpretation, analysis, extraction and/or transformation of received signals and information, such as template matching and/or online template matching.

Data acquisition device 114 may therefore assist with accurately detecting health data such as heart rate (HR) and heart rate variability (HRV), and relative to ECG-based techniques, data acquisition device 114 may function adequately with less precise placement. Moreover, contrary to ECG-based techniques, techniques disclosed herein may not need expensive conductive yarns or electrodes that need to contact the skin, or disposable contact electrodes which require precise placement, can cause skin irritation, are painful to remove, and require frequent reapplication. For ECG based techniques, placement of electrodes may also require shaving skin or uncomfortably pressing the electrodes into the skin.

Therefore, use of data acquisition techniques as described herein may allow for relatively easy and cost-effective measurement and derivation of physiological and behavioural data relating to a large group of users.

Such data may be particularly useful in combination with genetic data relating to the same users. Data acquisition facility 112 may be a genetic testing facility configured to analyze a sample from a user to obtain genetic data, such as gene polymorphisms. Genetic data may include, without limitation, sequencing of genes, genome, telomere length data and epigenetic testing data. For example, genetic data acquisition facility 112 may receive a sample of genetic material from user 116, such as a saliva sample, blood sample, tissue sample or the like. Genetic data acquisition facility 112 may be configured for performing at least a partial nucleotide (genetic) sequence using the obtained sample, thereby acquiring genetic data at particular points of interest in the genome. Genetic features such as single nucleotide polymorphisms (SNPs), insertions, deletions, recombinations, telomere length, methylation patterns and the like sampled at genetic data acquisition facility 112 may be those scientifically determined to be correlated with particular traits (e.g. phenotypes) of interest. In an example, genetic data testing facility 112 obtains measurements of telomere lengths in the DNA of user 116, which have been shown to be correlated to aging. Alternatively or additionally, traits such as activity and sleep patterns may be obtained from a personal data acquisition device and used to create population groups or bins, which may then be sampled for genetic data. Alternatively or additionally, traits can be defined based on groupings of genetic data and data from a wearable biosensor, and more complete genetic data may be obtained from individuals belonging to such groupings. For example, a grouping may be defined based on genetic factors such as long telomere length and health data from a data acquisition device, such as abnormally low sleep. Members of that grouping may be sampled for additional genetic data to search for previously-unknown genetic associations. Each server 102 includes a processor 118, memory 120, persistent storage 122, network interface 124 and input/output (I/O) interface 126.

Processor 118 may be an Intel or AMD x86 or x64, PowerPC, ARM processor, or the like. Processor 118 may operate under control of software loaded in memory 120. Network interface 124 connects server 102 to network 106. I/O interface 126 connects server 102 to storage 104 and may further connect server 102 to one or more peripherals such as keyboards, mice, USB devices, disc drives, and the like.

Software may be loaded onto server 102 from peripheral devices or from network 106. Such software may be executed using processor 118.

FIG. 3 depicts a simplified arrangement of software at a server 102. The software may include an operating system 128 and application software. The application software may include a database management system 130 and an analytics engine 132. Database management system may be a system configured for compatibility with the relational database model using a language such as SQL. Analytics engine 132 is configured to analyze data stored in storage 104, for example, to identify correlations between data of different types and to identify user actions predicted to be associated with desired health outcomes.

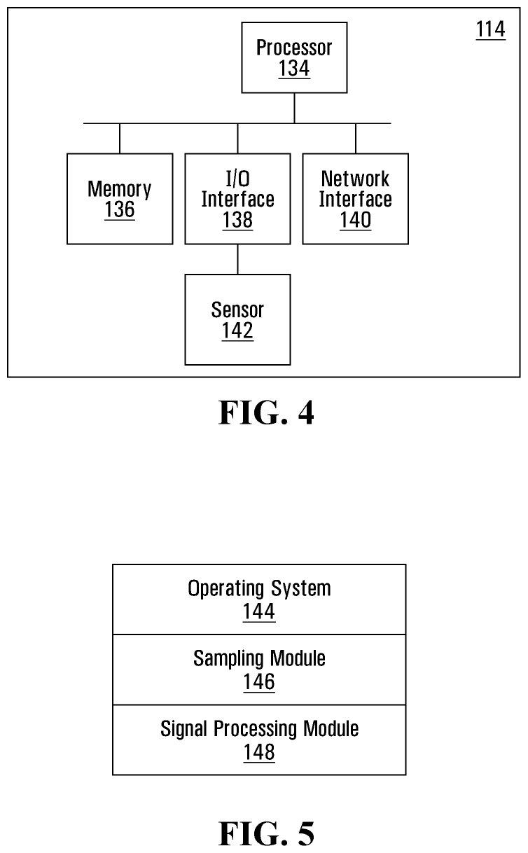

FIG. 4 is a block schematic diagram of components of data acquisition device 114. As depicted, each server 102 includes a processor 134, memory 136, persistent storage 138, communications interface 140 and input/output (I/O) interface 142.

Processor 134 may be an Intel or AMD x86 or x64, PowerPC, ARM processor, or the like. Processor 134 may operate under control of software loaded in memory 136. Processor 134 may be configured to apply various algorithms, filtering, and/or other techniques in processing and/or transforming received signals and/or derived features. Further, the processor 134 may also be configured to operate in one or more modes, and the selection of modes may be automatic, semi-automatic, or manual, depending on various factors (e.g., presence of a nearby computing device for pairing, battery level, whether a wearer is moving, signal noise, presence of artifacts, determined activity levels).

Communications interface 140 connects data acquisition device 114 to other devices such as client computing device 110 or server 102. I/O interface 142 may be further interconnected with one or more sensors 142 for acquiring measurements of user 116. In the depicted embodiment, sensor 142 is an accelerometer configured to output signals indicative of acceleration in three dimensions. However, other types of sensors are possible, such as optical sensors, temperature sensors, microphones, gyroscopes and the like.

Software may be loaded onto data acquisition device 114 from peripheral devices or from network 106. Such software may be executed using processor 134.

FIG. 5 depicts a simplified arrangement of software at data acquisition device 114. The software may include an operating system 144 and application software. The application software may include a sampling module 146 and a signal processing module 148. Sampling module 146 is configured to receive signals from sensor 142 indicative of measured values, and to provide the signals to signal processing module 148. Signal processing module 148 is configured to receive data from sampling module 146 and analyze the data to produce models or representations of, for example, user physiological or behavioural activity.

Physiological activity measured by data acquisition device 114 may include, for example, cardiopulmonary activity, bodily movements (e.g., step counts, bodily positioning, changes in orientation, changes in altitude, bodily acceleration, rotation, translation), positional changes, etc. In the context of this disclosure, the term cardiopulmonary may describe aspects related to the cardiovascular system (e.g., blood, heart, blood vessels, heart beats, heart murmurs, irregular blood flow, heart beat variations, heart rate), and/or the pulmonary system (lungs, respiration, trachea, bronchi, bronchioles, diaphragm, alveoli, among others). The term cardiopulmonary means one or both of cardiovascular or pulmonary.

Sampling module 146 and sensor 142 of data acquisition device 114 may be utilized, to obtain waveform (e.g., seismocardiography (SCG) readings. As will be apparent, raw waveform data obtained by sensor 142 may not be directly representative of activity of interest. For example, a waveform produced by chest movement may result from the combination of movements of different types, such as rising and falling of the chest due to inhalation and exhalation, as well as larger body movements, e.g., walking.

Accordingly, signal processing module 148 may be configured for application of various algorithms that may aid in the processing, extraction, and/or signal cleaning required to obtain (e.g. isolate) the waveform (e.g., SCG) readings of interest.

Data acquisition device 114 may be adapted for contact with bodily part(s) of user 116 (e.g., the chest, sternum, or rib cage) for efficient and/or accurate acquisition of various biological cues, signals, activities, etc. The apparatus may be in direct and/or indirect contact with the body, for example, through a garment, a positioning device 108 or the like, such that data acquisition device moves along with the user's body. In particular, data acquisition device 114 is held in direct or indirect contact such that mechanical microvibrations caused by the user's body are conducted to and detected by data acquisition device 114. For example, data acquisition device 114 may be coupled to the body by a garment, held against the body manually, or held in a bag, provided the bag is capable of transmitting mechanical vibrations to data acquisition device 114.

In some embodiments, data acquisition device 114 may be biased and/or affixed to engage and/or couple with the wearer's body. For example, data acquisition device 114 may rest against the body under the influence of gravity or may be urged against the body by the garment. In some embodiments, data acquisition device 114 may “hang” on the garment and/or otherwise be positioned “in” part of the garment (e.g., a pocket, a pouch), such that various signals may be measured (e.g., accelerometer signals) and various waveforms/features may be extracted (e.g., heart rate, respiratory rate). In some embodiments, data acquisition device 114 may be held against or in proximity to the body of user 116. For example, data acquisition device 114 may be held on a vest worn by user 116 or held in a bag such as a handbag.

In an example, data acquisition device 114 is a wirelessly enabled (e.g., using Bluetooth communications) small ˜30 mm or smaller circular device, ˜7 mm or less in height containing a micro-electro-mechanical system (MEMS) accelerometer (e.g., a 12-bit accelerometer or 14-bit accelerometer). Other designs and technologies are possible, and may be desirable in various circumstances. For example, in some embodiments, the sensor 142 may include a piezoelectric sensor, which may be adapted to track and/or monitor various signals based on pressure being applied to the sensor 142. In some further embodiments, the sensor 142 may include both a MEMS accelerometer and a piezoelectric sensor.

Thus, sensor 142 of data acquisition device 114 produces an output signal indicative of motion of the body of user 116, or a part thereof. For example, data acquisition 114 may be positioned against a wearer's chest and produce acceleration traces indicative of acceleration in up to three dimensions.

Raw data produced by sensor 142 may be recorded by sampling module 146 and processed by signal processing module 148 using analytical models to produce values representative of basic physiological activity, such as respiration rate, heart rate, respiration rate variability, heart rate variability and the like, and basic behavioural activity, such as position changes, steps taken and the like.

Signal processing module 148 may further analyze the basic physiological or behavioural data to derive more complex representations of physiological or behavioural activity. For example, signal processing module 148 may use values such as respiration rate, heart rate, respiration rate variability and heart rate variability to derive values representative of a sleep state or sleep quality of the user, exercise level or quantity of a user, or the like.

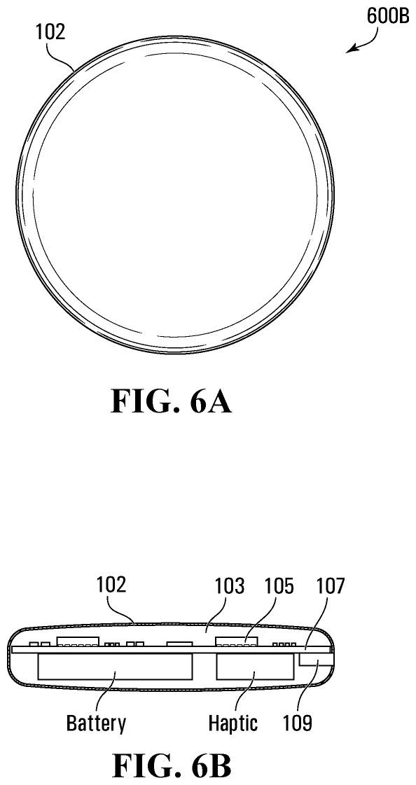

Data acquisition device 114 may be provided in a relatively small size for comfort and light weight. For example, data acquisition device 114 may have a small, generally cylindrical housing 150, as depicted in FIGS. 6A-6B, 7A-7B and 8A-8B. In the depicted embodiment, housing 150 contains a printed circuit board (PCB) 152 and components of data acquisition device 114 are formed as integrated circuit dies 154 carried on circuit board 152. Sensor 142 is connected with PCB 152 in communication with dies 154.

In an example, housing 150 measures 7 mm in thickness and 32 mm in diameter and has outer edges with a 2 mm fillet. A microUSB plug opening may be provided somewhere in the midsection or just beneath the 3.5 mm mark.

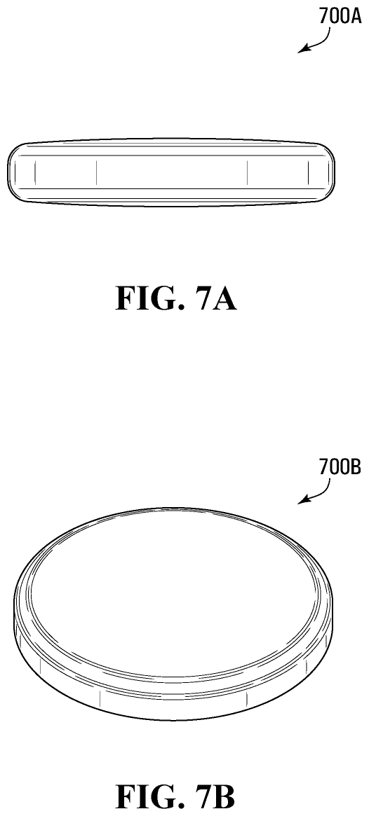

FIG. 7B is a sample perspective view of data acquisition device 114, according to some embodiments. As illustrated the top face the logo (highlighted) may be indented by 0.5 mm.

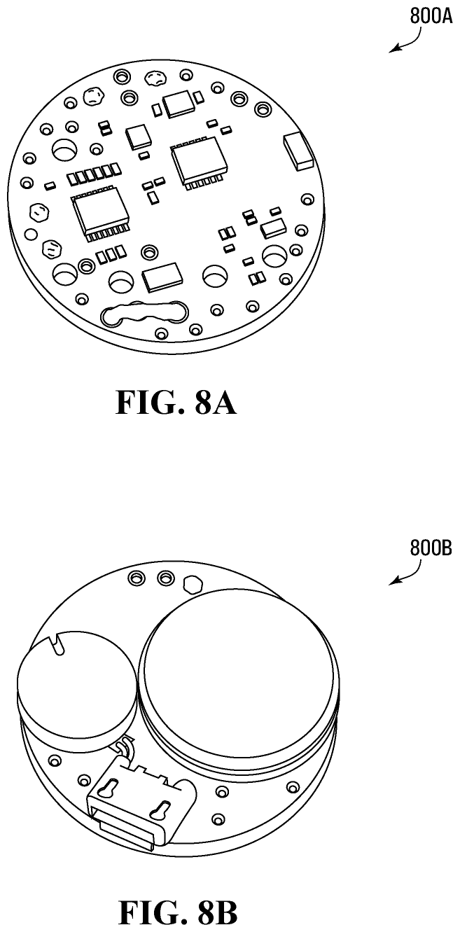

FIG. 8A is a perspective view of a printed circuit board, according to some embodiments. As depicted, the diameter of the PCB may be 30 mm.

FIG. 8B is a perspective view of a printed circuit board, according to some embodiments. As depicted in FIG. 8B, there may be various components, including a Lir 2032 battery, haptic motor, and microUSB, among others. In some embodiments, the Lir 2032 battery may have solder tabs that are connected to the PCB.

Sensor 142 (e.g., a single accelerometer) is operable to produce a data stream indicative of movements of a wearer's chest. There may be one or multiple sensors 142, and the sensors 142 may be independent or configured to interoperate with one another. For example, the readings from multiple sensors 142 may be aggregated, combined, and/or otherwise transformed (e.g., through digital signal processing) such that features may be more readily identified, noise may be removed, analyses may be verified, etc. The one or more sensors 142 may be analog, digital, and there may be multiples of the same type of sensors 142, different sensors 142 provided, or both.

As an example where a second accelerometer is used, the two accelerometers may be located at different positions and the information from the first and second accelerometers may be utilized in combination, for example, by finding differences between the signals to aid in detecting and/or ultimately adapting for motion artifacts. Such an approach may be useful in finding heart rate in ambulatory conditions, but may require further onboard processing power, as well as higher battery consumption.

In some embodiments, the sensor 142 may be operable to produce a data stream 114 by sampling at a first frequency and said processor 110 may be configured to decimate said signal to a second frequency lower than said first frequency. The sensor 142, in some embodiments, may be one or more MEMS accelerometers.

Variations are possible. For example, a sensor 142 (e.g., an accelerometer) may be provided where the sensor is able to acquire a signal at a frequency (e.g., 800 Hz) and decimates in the sensor (e.g., the accelerometer), outputting a signal at a desired frequency (e.g., 200 Hz), where, for example, the processor may need a signal at the desired frequency (e.g., 200 Hz). In some embodiments, there may be decimation for signals associated with, for example, an activity and respiration to another frequency (e.g., 25 Hz).

In some embodiments, the sensor 142 may be a system on a chip (SoC), and accordingly, the sensor 142 may have various on-board capabilities to be able to perform various types of computing functions, such as pre-processing, decimation, among others. For example, an accelerometer may be provided as a SoC, which may have its own processor, and the accelerometer and/or its onboard components may be able to perform decimation prior to processing by the processor 134.

In an example, a single MEMS accelerometer may be used for simplicity and convenience. For example, a single MEMS accelerometer may be affordable and may facilitate efficient extraction of activity level and classification, heart rate, heart rate variability, and respiration rate, captured with a single data acquisition device 114.

In some examples, the accelerometer has at least 1 mg/digit sensitivity for accurate heart rate, and respiration rate measurement, due to the small amplitudes of the two signals. At least a 12 bit, 2 g accelerometer may be used.

In some embodiments, the sensors 142 may include a gyroscope and an accelerometer. Signals acquired from these sensors 142, may be used, for example, in determining the Euler angle, and a Kalman filter may be applied for noise reduction. Such an approach may, for example, be utilized to determine ambulatory respiration, and a similar method may be possible for heart rate estimation as well by using a Kalman filter to remove noise from ambulatory heart rate signal.

Data acquisition device 114 may be coupled to the body of user 116 by a positioning device. In particular, data acquisition device 114 may be adapted for contact with bodily part(s) of a wearer (e.g., the chest, sternum, or rib cage) for efficient and/or accurate acquisition of various biological cues, signals, activities, etc. The apparatus may be in direct and/or indirect contact with the body, for example, through a garment, a positioning device 108, etc., and in some embodiments, may be biased and/or affixed to engage and/or couple with the wearer's body. For example, the data acquisition device 114 may be biased to be proximate the body through gravity, a biasing device, biasing means (e.g., a specially configured shirt having elastic materials), among others. The data acquisition device 114, in some embodiments, may “hang” on the garment and/or otherwise be positioned “in” part of the garment (e.g., a pocket, a pouch).

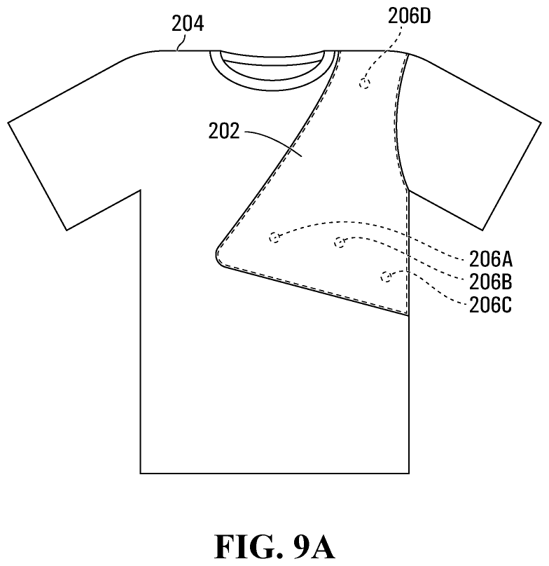

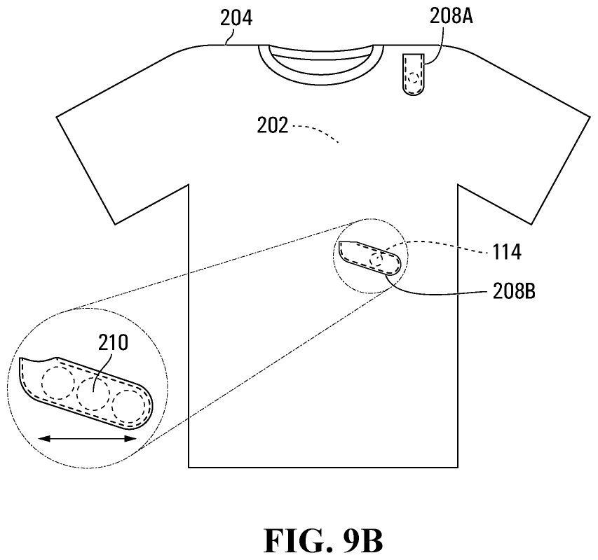

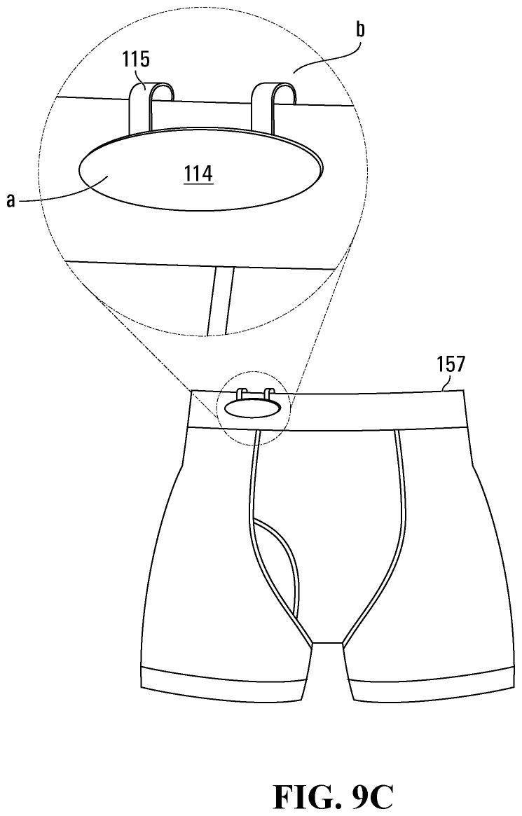

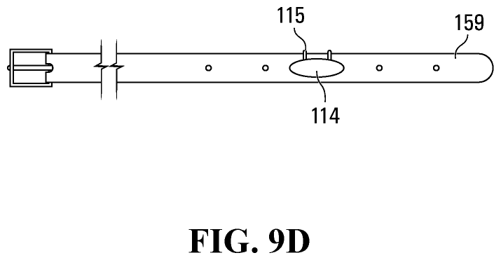

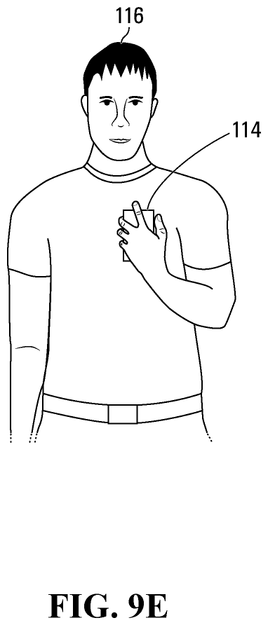

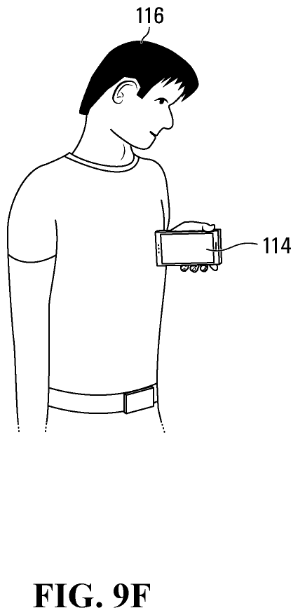

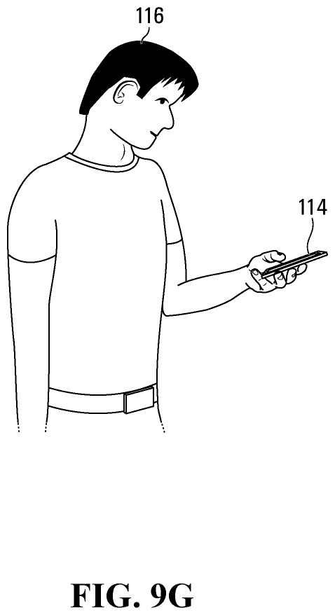

FIG. 9A is a front view of a garment 204 which can serve as a positioning device. FIG. 9A further illustrates an anatomical zone/region of data acquisition 202, as well as potential placement locations 206A . . . D of data acquisition device 114 in various embodiments. The potential placement locations 206A . . . D may indicate where the positioning device 156 may be positioned proximate to (e.g., in, around, attached to, extending from) a pocket formed in a garment 204 that may be located near and/or on potential placement locations 206A . . . D.

In some embodiments, the positioning device 156 may not necessarily be a pocket, and may be other types of affixing and/or positioning components, such as a magnetic attachment, or a clip (e.g., to bra).

The garment 204 may be a shirt, in some embodiments. For example, the device may be placed into an interior pocket into a t-shirt located at the point of maximal impulse on the chest such that the device rests against this location of the chest. Other bodily landmarks may be considered, such as a rib, the sternum, a collarbone, a solar plexus, etc. While a t-shirt is shown, the garment 204 is not limited to a t-shirt and may be a tank top or any other garment.

FIG. 9B is a front view of a garment 204 illustrative of potential placement positions 208A, 208B for a pocket for holding data acquisition device 114, according to some embodiments. Potential locations of pockets may be on the inside of the shirt, and data acquisition device 114 may be stored inside a pouch 210. In some embodiments, the pouch 210 may be configured such that the device can be placed at various locations (as denoted by 1, 2, and 3) and the orientation within the pouch 210 may be determined based on whichever is best suited for the location (e.g., vertical, diagonal).

Sensor 142 may be fixed to the torso of the wearer, which may for example, provide more accurate activity levels and body position levels relative to a conventional wrist-based wearable fitness tracker.

In some embodiments, data acquisition device 114 may be located on a wearer such that data acquisition device 114 rests at a point of maximal impulse, which may, for example, allow for a more accurate capture of activity (fixed to the torso vs. the wrist), the capture of a respiration rate, and heart rate, as well as heart rate variability (heart rate variability). Capturing the heart rate variability may present challenges as noise and missed beats may have a significant effect that may alter the power of the frequency domain, significantly skewing the power spectral domain analysis.

Data acquisition device 114 may be coupled to the body of user 116 by a positioning device 156. Positioning device 156 may be operable for holding the sensor 142 proximate an anatomical landmark on said wearer's chest. In some embodiments, the positioning device 156 is configured to facilitate loose and/or light coupling (e.g., light contact) between the sensor 142 and the wearer's chest. For example, the positioning device 156 may be a relatively loose-fitting shirt or other suitable garment. The sensor 142 may, for example, hang from the fabric of a garment, and the signal (e.g. chest movements of the wearer) may be conducted through the fabric of the garment, etc. In some embodiments, positioning device 156 may be a shirt that fits a wearer's body sufficiently tightly to maintain mechanical contact between sensor 142 and the body, such that sensor 142 moves along with the wearer's chest, without exerting an uncomfortable amount of compressive pressure.

Other methods of mounting and positions of data acquisition device 114 are possible. For example, data acquisition device 114 may be placed on a user's belt, in a pocket such as a breast or pant pocket, carried in a bag, or the like.

FIGS. 9C-9D depict examples of a data acquisition device 114 mounted to clothing of a user 116. FIG. 9C depicts data acquisition device mounted to a pair of shorts 157. Shorts 157 may be an undergarment. FIG. 9D depicts data acquisition device 114 mounted to a belt 159. As depicted, data acquisition device 114 has clips 115 which act as a positioning device. Clips 115 engage part of an article of clothing, e.g. shorts 157 or belt 159, to secure data acquisition device 114 thereto. Data acquisition device 114 may be positioned such that it faces the user 116, in which case data acquisition device 114 may contact the user's body. Alternatively, data acquisition device may not contact the user's body, and vibrations may be conducted to the data acquisition device 114 by way of the garment and clips 115. For example, the data acquisition device 114 may face away from user 116. Clips 115 may be formed integrally with a housing of data acquisition device, or may be attached by any suitable method.

Suitable positions and mounting methods include those which provide a path for mechanical vibrations associated with a user's heart beat or respiratory activity to be conducted from the body of user 116 to data acquisition device 114. For example, data acquisition device 114 may be carried in a bag, provided the bag is sufficiently rigid to conduct vibrations. Preferably, data acquisition device 114 is mounted close to the chest of user 116.

In some embodiments, user 116 may hold data acquisition device 114 against user 116's body, e.g. against the chest. For example, FIG. 9E depicts a schematic view of a user 116 holding a data acquisition device 114 against the chest of user 116. Data acquisition device may capture user data such as heart rate, respiration rate, respiration rate variability or the like, as described herein. FIG. 9F depicts another example wherein a user 116 holds data acquisition device 114 with a bottom side contacting the chest. FIG. 9G depicts another example wherein user 116 holds data acquisition device in one or both hands, without physically contacting the chest.

As depicted in FIGS. 9E-9G, data acquisition device 114 has a display. For example, data acquisition device may be a smartphone with an integrated accelerometer and a display. At least some processing of acquired signals may be performed on data acquisition device 114 and feedback may be provided to user 116. Conveniently, feedback may be shown on the display and user 116 may position data acquisition device 114 in a position for viewing the screen while acquiring data. Thus, user 116 may receive feedback quickly (e.g., substantially instantaneously) after measurement of data with data acquisition device 114. Feedback may be provided, for example, by way of a mobile application, which may use measured data such as heart rate and respiration rate data for directing exercises related to meditation or mindfulness.

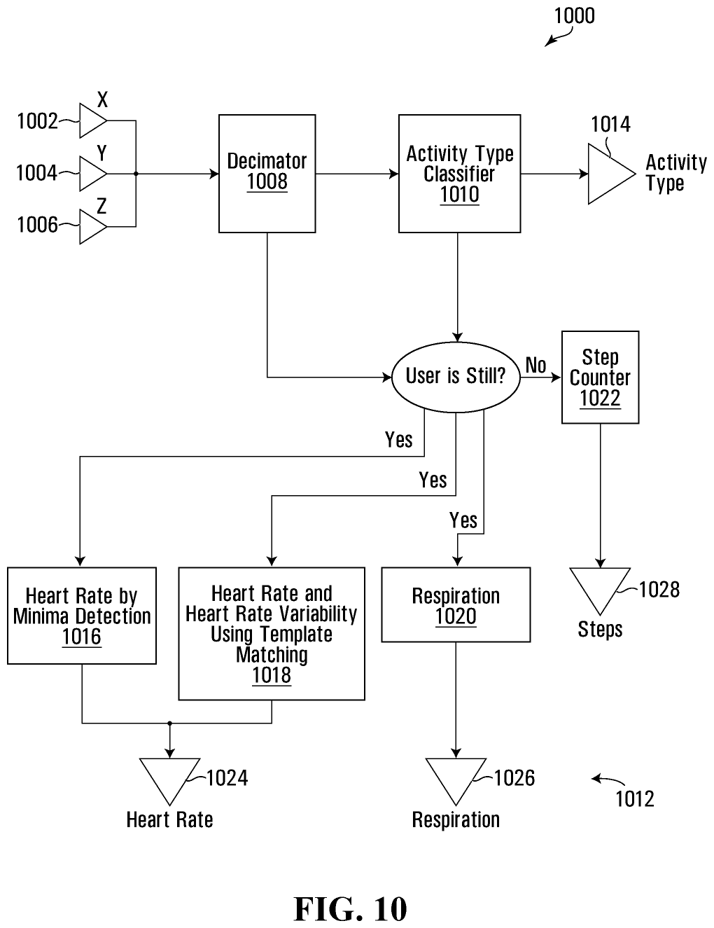

FIG. 10 is a workflow diagram that illustrates the usage of processes to measure cardiopulmonary data of a wearer, according to some embodiments.

The processes include various components, such as a decimator, an activity type classifier, a step counter, a heart rate detector, and a respiration detector. Such components may be software modules forming part of acquisition module 146 or signal processing module 148 (FIG. 5). Alternatively, one or more of the components may be formed as a discrete hardware unit.

The classifier may be used to indicate whether the wearer is still. If so, information from sensor 142 may further be used to calculate, for example, the user's heart rate, heart rate variability, respiration rate or respiration rate variability. In some embodiments, if the wearer is not still, a step counter may be used to determine the number of steps taken by the wearer, etc. The steps counted by the step counter, for example, may be utilized in various determinations associated with the activity type of a wearer.

Various de-trending techniques may be utilized to process the received data and/or extracted data to indicate, for example, only the absolute changes in values. In some embodiments, data acquisition device 114 is configured to detrend the HRV instantaneous heart rate signal, detrending raw signals with high pass filters provided for RR, HR, and template matching algorithms.

Various filters can be applied, such as infinite impulse response (IIR) and finite impulse response (FIR) filters selected based on a more efficient filter for a particular use case. Filters may be analog or digital filters and may be implemented in software, hardware or a combination thereof. Filters may, for example, be implemented in specialized processing circuitry.

In some embodiments, principal component analysis techniques (PCA) may also be applied, for example, PCA may be applied to find the significant signal in a multivariate signal. In sample experimental results, Applicants found that the z-axis contained over 95% of the signal weight, and the other two axes were not as useful for respiration and heart rate calculation. Eigenvalues may be calculated as the weights for each axis that are derived from using PCA. For example, the eigenvalues may be 0.01 for X, 0.04 for Y and 0.95 for Z.

The processor 134 may be configured to set a number of beats to establish an adaptive threshold (e.g., an average of 8 beats and 40% for a sample threshold) to determine a general trend. Such an approach may be based on a consideration that beats close to each other in time may be similar to each other. Further, on an embedded system, for performance reasons, the mean may also be determined through performing a bit shift operation on the sum of the samples.

Applicants analyzed experimental results on collected samples having a range of thresholds from 65% to 25%, and found that a 40% threshold resulted in most ectopic beat rejection, which maintaining a very low error in detecting the correct beats.

In some embodiments, the processor 134 may be configured to determine instantaneous heart rate from 2 consecutive beats. However, instantaneous HR may vary quite significantly beat-to-beat (e.g., as the instantaneous heart rate tends to oscillate with each inspiration and expiration), and monitoring a period of at least 5 seconds may facilitate determining a mean period which may be closer to the mean over 60 seconds.

For example, while in high intensity activity or when the heart rate is high, the beat-to-beat interval changes are minimal; therefore the processor 134 may only need to determine 3-5 beats to accurately predict a heart rate.

In some embodiments, the processor 134 may be configured to perform various determinations and/or calculations using fixed point operations, in contrast to floating point operations. For example, in low power microcontrollers without floating point units, performing floating point operations may either not be supported or may need to be expensively emulated. Accordingly, processor 134 may be configured to convert floating point numbers into fixed point numbers (e.g., q15 format by multiplication with a factor of 2\hat{ }15).

Fixed point calculations may be generally faster even with the presence of an FPU, and in some embodiments, any calculations that can be conducted in fixed point reasonably may be performed in fixed point. The advantage is in chips that support hardware level digital signal processing (DSP), including vector processing, and FIR filter capabilities, allowing for expeditious fast Fourier transforms (FFTs), convolutions, cross-correlations, filtering, and vector operations.

Using fixed point calculations, digital filters may also be applied to perform single cycle adds, multiples, and bit shifts, which may be computationally less intensive relative to floating point calculations.

While fixed point filters may be prone to quantization noise, some embodiments may include the usage of noise shaping to mitigate some of the effects of quantization noise. Similarly, signal saturation may be eliminated by careful gain-staging. As a non-limiting example, to prevent the saturation of the signal, data acquisition device 114 may be configured to only scale up by a power of 2, where the highest possible value, multiplied by the gain factor, maintains the signal below the highest possible value available. In the case of a signed 16 bit integer, that would be 32767.

Conversely, if data acquisition device 114 was configured to utilize a q15 format where an accelerometer value from +2 g to −2 g is multiplied by 2\hat{ }15 to use the full precision afforded by a 16 bit signed integer, and the filters had a gain factor greater than 0 dB, the signal may saturate at higher input amplitudes (i.e. at higher accelerometer values).

### Decimation

As indicated in the flowchart 1000, acceleration signal inputs, in the X-axis 1002, Y-axis 1004, and Z-axis 1006, may be sampled (e.g., acquired) at a particular frequency (in this example, 800 Hz is selected but other values are possible). These signals may optionally be provided to a decimator at block 1008, which decimates the signal (e.g., to 200 Hz) and, in some embodiments, may apply an anti-aliasing filter (e.g., with a cut off frequency of 50 Hz). The decimated signal (e.g., a 200 Hz signal) may be used for heart rate/heart rate variability measurement.

Decimation may be performed, for example, if a sensor or processor cannot process data at the frequency at which acceleration data is sampled, or if data is not required at the high sampling frequency. Decimation may reduce operations performed by processors, which may in turn reduce power consumption and increase battery life.

The signal may be further decimated to 25 Hz with an anti-aliasing filter at 10 Hz, and the 25 Hz signal is used for energy expenditure calculation by an activity type classifier at block 1010 to determine activity type and to trigger the respiration and heart rate algorithms. The signal may also be used by the respiration algorithm.

In an alternative embodiment, data acquisition device 114 may instead acquire acceleration signal inputs at a lower frequency to perform energy expenditure and/or respiration analysis as a default setting, and when the processor 134 determines that the wearer is still, the processor 134 may then cause the acquisition of the signal at a higher frequency (e.g., 800 Hz) for other types of signal tracking and/or >3 Hz for activity tracking. In other words, an alternative approach may include instead of starting acquisition at a high frequency, data acquisition device 114 can also start low or using sensor (e.g., accelerometer) interrupts based on a predetermined thresholds for free-fall, motion, orientation, among others.

If the processor 134 starts signal acquisition at a low frequency, then an algorithm may be conducted as illustrated and described in accordance with other embodiments.

### Activity Classification

Placement of data acquisition device 114 on the body core facilitates the calculation of activity type with high accuracy. At block 1010 signals from sensor 142 are used to classify the activity level of user 116.

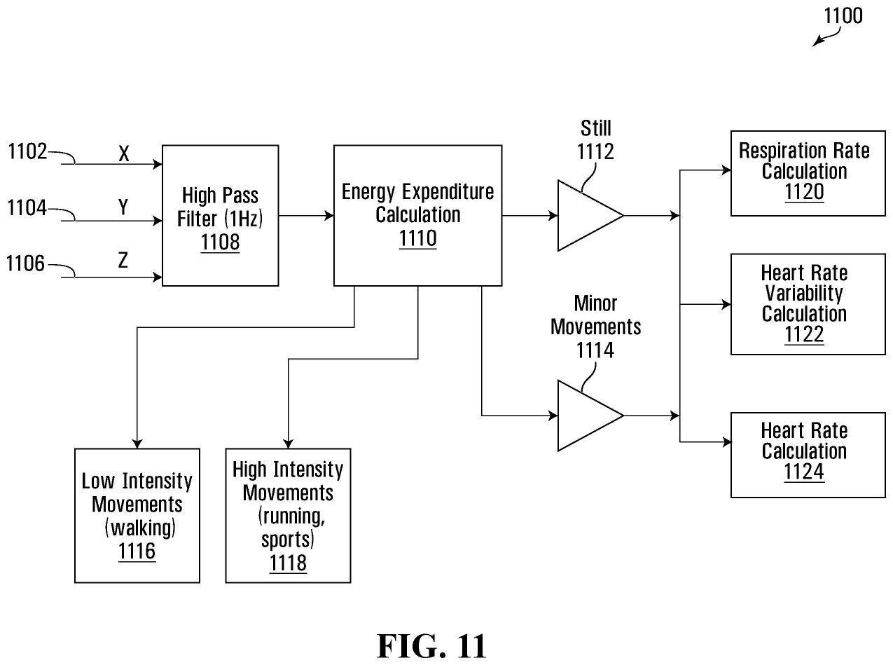

An example process of activity level classification is depicted in FIG. 11. As depicted in FIG. 11, the sensor inputs in the X, Y, and Z axes are provided into a high pass filter 1108. After applying the high pass filter, an approximation of energy expenditure is calculated at block 1110. The approximation may be calculated by taking the mean of the integration of the x, y, z axis signals within a time interval, i.e., sum(sqrt((x(i+1)−x(i))\hat{ }2+(y(i+1)−y(i))\hat{ }2+(z(i+1)−z(i))\hat{ }2), where i and i+1 denote measurements at consecutive sampling intervals. Other types of calculations may be performed, and the integration of the signals is provided as a non-limiting example.

Based on the level of energy expenditure, a classification may be made to determine whether the movement can be classified as “still”, still with minor movements (like rocking in chair etc.), low intensity movement (like walking), and/or high intensity movement (like running, sports) 1118.

If current activity state is still, then a calculation may be made to determine the respiration rate at block 1120, the heart rate at block 1122, and heart rate variability at block 1124. When the activity state is minor movements a calculation may be made in relation to heart rate 1124 but not respiration rate 1120 or heart rate variability 1122. Optionally, the apparatus may be configured to turn off the algorithms if activity is in other states (high and low intensity movement).

### Respiration Rate Detection

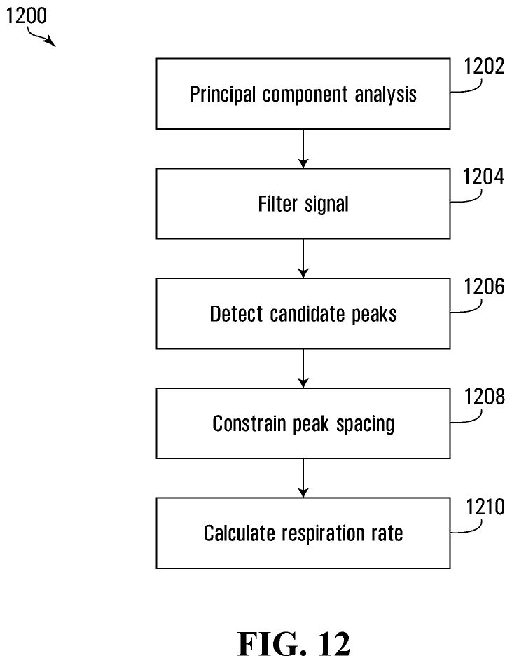

At block 1016, respiration rate detection is performed. FIG. 12 depicts an example process of respiration rate detection. At block 1202, principal component analysis is performed using the signal (e.g., 25 Hz) decimated from raw acceleration signal (e.g., 800 Hz) to determine the principle acceleration signal. At block 1204, a band-pass filter may be applied (e.g., between 0.1 Hz and 1.0 Hz, or 0.2 and 0.6 Hz among others in similar frequency ranges) to the acceleration signal.

At block 1206, peak (maxima and minima) detection may be performed on the decimated data using adaptive thresholding. For example, an adaptive threshold may be calculated based on a number (e.g., 8) of previous samples. The mean of the previous samples may be calculated and the threshold may be defined based on an allowed deviation (e.g., of 40%) from the mean of the previous samples. Minima and maxima may be defined as points with values deviating from the mean by more than the threshold value.

Each pair of a maximum and a following minimum correspond to one breath cycle. At block 1208, detection of minima and maxima are constrained such that each pair of detected Maxima/Minima are spaced (e.g., at least 500 ms) away from one another.

At block 1210, a respiration rate calculation is performed wherein the total breath cycles/*60 seconds is the determined as the breaths per minute.

### Respiration Algorithm Using Autocorrelation

Alternatively, respiration rate may be detected at block 1016 using autocorrelation. Specifically, principal component analysis may be performed on the decimated (e.g., 25 Hz) signal to find the principal acceleration signal. A filter (e.g., a band pass filter between 0.1 Hz and 0.7 Hz, or 0.2 and 0.6 Hz among others in similar frequency ranges) may be applied to the acceleration signal. Autocorrelation of a duration (e.g., 15 seconds) of the previous filtered signal may be performed, and the inverse of the period of the autocorrelation signal may be determined as the respiration rate. That is, the filtered signal may be correlated against itself at different points in time, over a window of a selected duration, such as 15 seconds. Such correlation may output a signal with evenly-spaced peaks. The interval between adjacent peaks corresponds to the average respiration rate interval during the window. Respiration rate may be calculated as the inverse of that interval.

### Heart Beat Detection

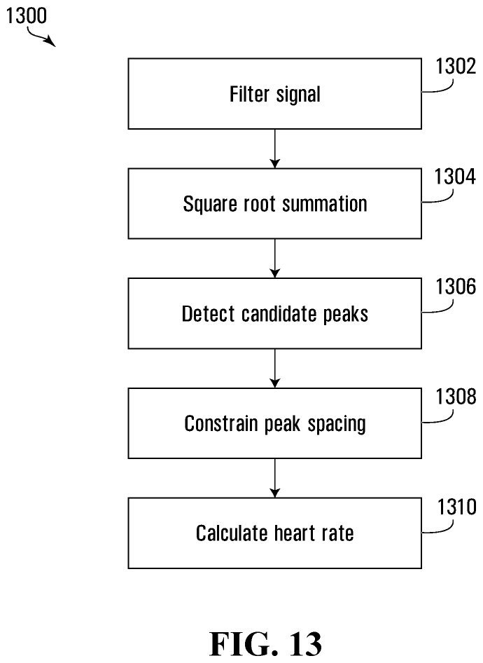

At block 1020, heart beat detection is performed. An example process of heart beat detection is depicted in FIG. 13. The process utilizes the raw acceleration signal measured by sensor 142 or the decimated signal (e.g., the >=200 Hz signal) produced at block 1008.

At block 1302, each of the x, y and z-axis raw or decimated signals may be filtered (e.g. using a band pass filter between 7 Hz and 13 Hz). Such filtering may isolate components of the signals associated with the heart beat of user 116. At block 1304, the filtered components may then be used to calculate the square root summation of the squared components of each signal.

At block 1306, candidate minima and/or maxima (individually and collectively referred to as peaks) may be found in the signal using an adaptive threshold algorithm, and in some embodiments, the adaptive threshold may be set at 40 percent of the mean of the previous 8 peaks. In other embodiments, the adaptive threshold may be anywhere between 40%-60% of the previous 8 peaks.

At block 1308, detection of peaks is constrained so that they are spaced apart by at least a threshold time. That is, a measurement may be identified as a peak only if it is below the adaptive threshold and it occurs at least a threshold time after the preceding peak. For example, peak may be required to be above the adaptive threshold and spaced at least 250 ms away from one another (max bpm of 240) may be allowed, and the apparatus may determine that peak to peak is a candidate peak to peak interval.

Optionally, prior to block 1304, a buffer of N peak to peak intervals may need to be completed without a noise event for a successful heart rate calculation. A noise event may be defined as an interval that is at least 25 percent off from the length of the previous interval. In some embodiments, the algorithm may require a buffer of N (where N may equal 8) peak to peak intervals completed to average for a Heart Rate while tolerating up to a threshold of a set number of noise events (where number of events may range from 1 to 4). The threshold for number of noise events is directly proportional to the frequency of heart rate measurements, and inversely proportional to the accuracy of each heart rate measured. If total number of noise events reaches the threshold for noise events before the peak to peak interval buffer is filled, the buffer and noise event count is reset, and will require N more peak to peak intervals with noise events less than the noise event threshold to calculate the heart rate.

At block 1310, the heart rate in beats per minute may be calculated by dividing sixty seconds by the mean of the buffered peak to peak intervals in seconds.

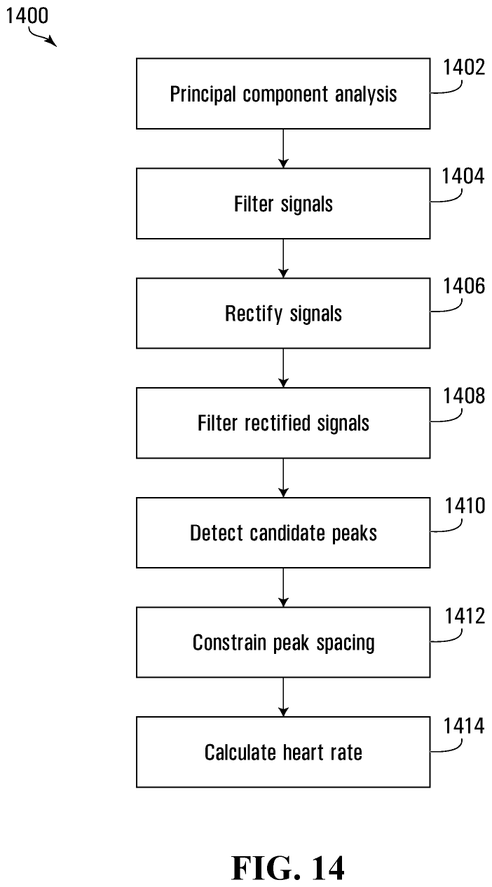

FIG. 14 depicts an alternative process for heart beat detection. At block 1402, principal component analysis is performed on the signal to obtain the principal acceleration signal. At block 1404, a filter, such as a band pass filter between 5 Hz and 50 Hz is applied. The roll, tilt and/or pitch angles of the accelerometer may be used to isolate heart rate signals. For example, roll, tilt and/or pitch angles of the accelerometer may be used in place of the raw acceleration values as described above. At block 1406, the filtered signal may be rectified to find the signal, and various filters may be applied.

Optionally, at block 1408, a low pass filter is applied to the rectified signal with a 8th order Butterworth filter (e.g., at 2 Hz), and a high pass filter is applied to the signal with a 4th order (e.g., 0.7 Hz) Butterworth filter to remove a DC offset.

At block 1410, candidate peaks (maxima and minima) may be found in the signal using an adaptive threshold algorithm, and in some embodiments, the adaptive threshold may be set at 40 percent of the mean of the previous 8 peaks.

At block 1412, detection of peaks may be constrained so that they are spaced apart by at least a threshold time. That is, in some embodiments, a measurement may be identified as a minimum only if it is below the adaptive threshold and it occurs at least a threshold time after the preceding minimum. For example, minima may be required to be below the adaptive threshold and spaced at least 250 ms away from one another (max bpm of 240) may be allowed, and the apparatus may determine that minima to minima is the heart rate interval. Periodically (e.g., every 15 seconds), the algorithm may include the removal of heart rate interval outliers where the deviation is greater than 25% of the mean of the heart rate interval. At block 1414, a heart rate is determined using the calculation heart rate=total(rrintervals)/sum(rrintervals)*60 or mean of the rr intervals/sum of rrIntervals*60.

Example PCG/SCG, Heart Beat Detection, Heart Rate Variability Detection

Based on peak detection, an algorithm may be utilized to estimate at where an S1-S2 pair may lie in a 5 second sample. Each of these pairs may then be labeled as candidate templates. Each candidate template is processed through the five second interval with a correlation detection algorithm, such as cross-correlation or cosine similarity, to find matches. The correlation signal for the template with the highest correlation scores may then be selected as the interval signal, and the distance between points of highest correlation was selected to be the various intervals (e.g., R-R intervals, S1-S2 sounds, aortic opening features, aortic opening intervals, among others). Applicants found that such an approach performed satisfactorily for noisy signals, as even if noise contamination was present, as if the S1 and S2 pair repeats in any fashion, a correlation peak was found at the point where S1 and S2 pair repeats. The buffer was then moved up to before 150 ms of the last correlation peak point, and another 5 seconds of data was assessed for R-R intervals.

Accordingly, a technique may be provided having a feature extraction algorithm (e.g., a beat detection algorithm to roughly classify S1-S2 pairs), a window size that may be optimized for performance with the algorithm (e.g., due to the changing nature of SCG signals, the SCG signal provides a high degree of temporal locality, but does change significantly over time and choosing an appropriate windowing length allows for higher accuracy), and a correlation algorithm (e.g., cross correlation or cosine similarity).

The template-matching algorithm relies on the features of both S1 and S2 sounds to accurately find the heart beat locations. These locations are significantly more resistant to noise than simple minima/peak detection with regards to the beat intervals, leading to robust and accurate HRV measurement from SCG signals through template matching. The algorithm is also significantly more resistant to noise, where the S1 and S2 have very little separation from the noise floor. This is therefore essential for any HRV related analysis that requires frequency domain analysis, which is very sensitive to noise.

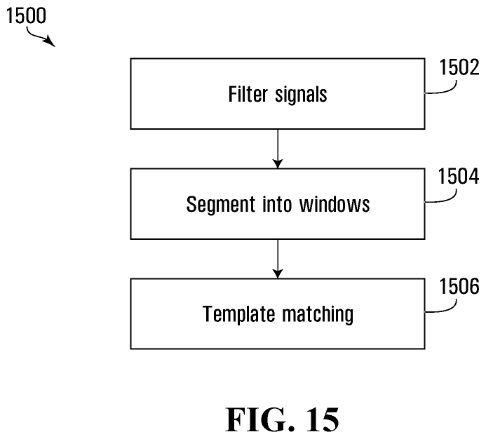

At block 1020, heart rate variability may also be determined. FIG. 15 depicts an example process of determining heart rate variability.

At block 1502, a 4th order Butterworth band pass filter between 10 Hz and 30 Hz may be utilized to isolate heart rate signals from the principal component acceleration signal. A low pass filter may be applied to the rectified signal with a 8th order Butterworth filter at 3 Hz to preserve the S1 and S2 peaks. A high pass may be applied to the signal with a 4th order 0.7 Hz Butterworth filter to remove the DC offset.

At block 1504, the signal may be segmented into a N second window. Maxima detection may be utilized to find S1, S2 heart sounds using automatic thresholding based on the previous 8 peaks and each S1-S2 pair spaced at least 250 ms away. If errant beats are found due to noise or there is a requirement for better start/end point detection of the S1/S2 sounds or the SNR is low, segments of signal that may contain S1 and S2 as potential templates are selected based on peak detection.

At block 1506, the N sized signal window is run through online template matching using cross correlation, cosine similarity or other correlation methods, and beats are selected based on a similarity signal, where the similarity exceeds an experimentally determined correlation threshold. The selected beats may be more noise resistant and will be closer to the start/end point of the feature being extracted than the peaks in a noisy signal.

Noise Resistant and Efficient Template Selection and Online Template Matching for Repeating Feature Detection in Biosignals

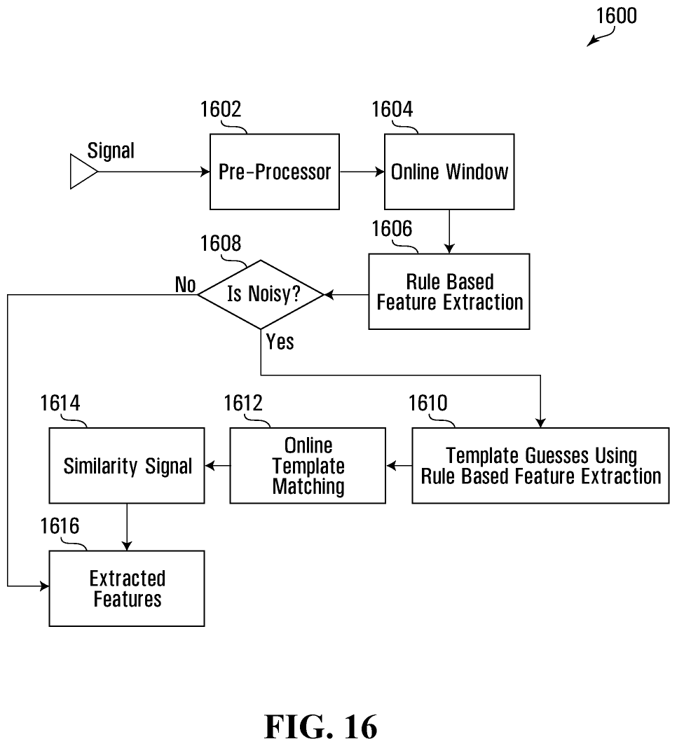

FIG. 16 is a workflow diagram illustrating an example process of template selection and matching.

There may be various steps of preprocessing 1602, applying an online window 1604, rules based feature extraction 1606, a determination of whether the signal is noisy 1608, among others. Based whether the signal was determined was noisy or not noisy, different paths may be selected, for example, if the signal is noisy, there may be steps of templating guesses using rule based feature extraction 1610, performing online template matching 1612, determining a similarity signal 1614 to obtain the extracted features 1616.

It should be noted that this algorithm is waveform agnostic and does not have to be from SCG (e.g., the waveform may be ECG, PCG, Photoplethysmography (PPG), and bioimpedance, electroencephalography (EEG), electromyography (EMG), electrooculography (EOG), actigraphy, among others, etc. . . . ). The algorithm may include preprocessing the signal using digital signal processing techniques such as filtering, transforms, etc. to extract (e.g., bring out) template features in the signal, and a rule based approach may be utilized. Example rules based approaches may include, for example, peak detection and thresholding for feature extraction because biosignals have repetitive cycles, features, etc.

The algorithm may further include determining noise amounts based on past collected features, and if the algorithm determines that the signal is noisy or there is a requirement for precise location of the feature start and/or end point, the algorithm may include the selection of templates with signal segments with best guess features from the rule based algorithm.

The algorithm may further include performing template matching using candidate templates using correlation methods (e.g., cross correlation, cosine similarity).

A template with the best performance based on previous features and similarity signal may be selected.

### Sample Signal Traces

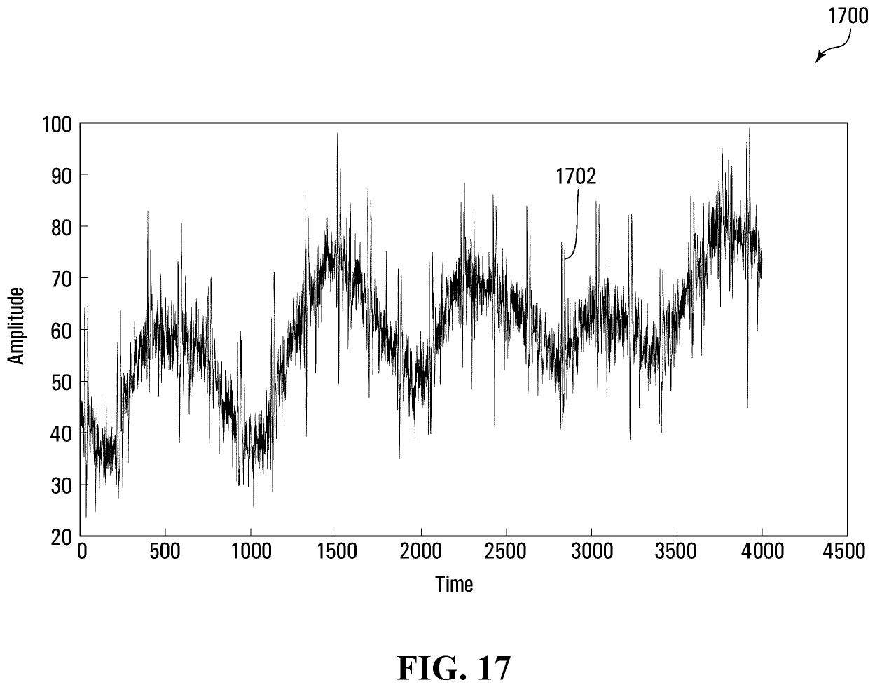

FIGS. 17-26 are traces of accelerometer amplitude against time, according to some embodiments.

FIG. 17 is a trace of an example raw Z Axis Signal from accelerometer 20 second sample, according to some embodiments. A trace is indicated as 1702.

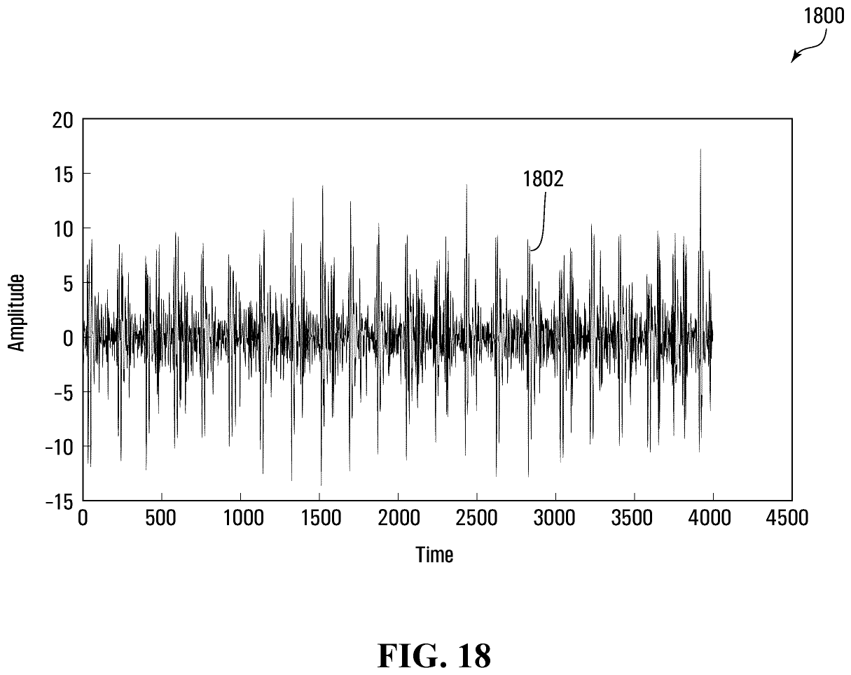

FIG. 18 is a trace of an acceleration signal Filtered for SCG with band pass of 10 Hz to 35 Hz, according to some embodiments. A trace is indicated as 1802.

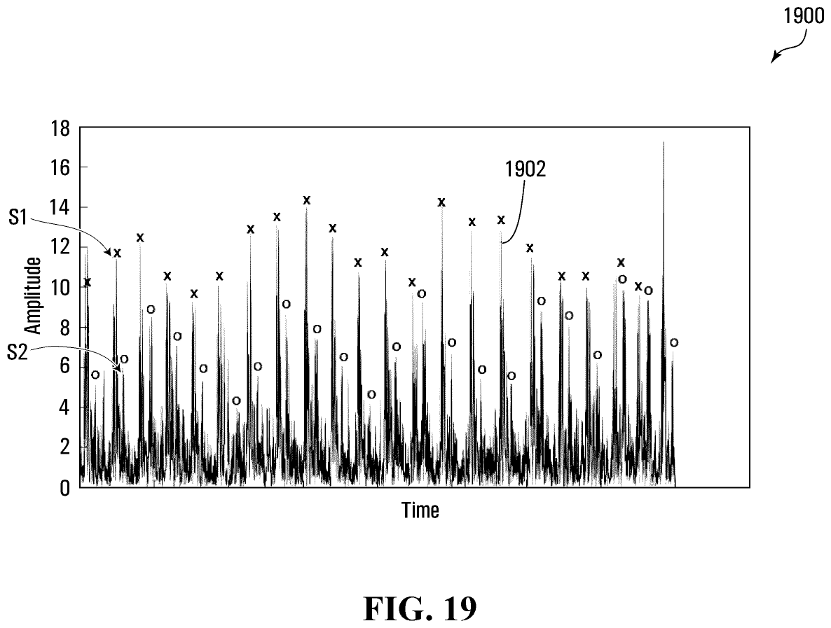

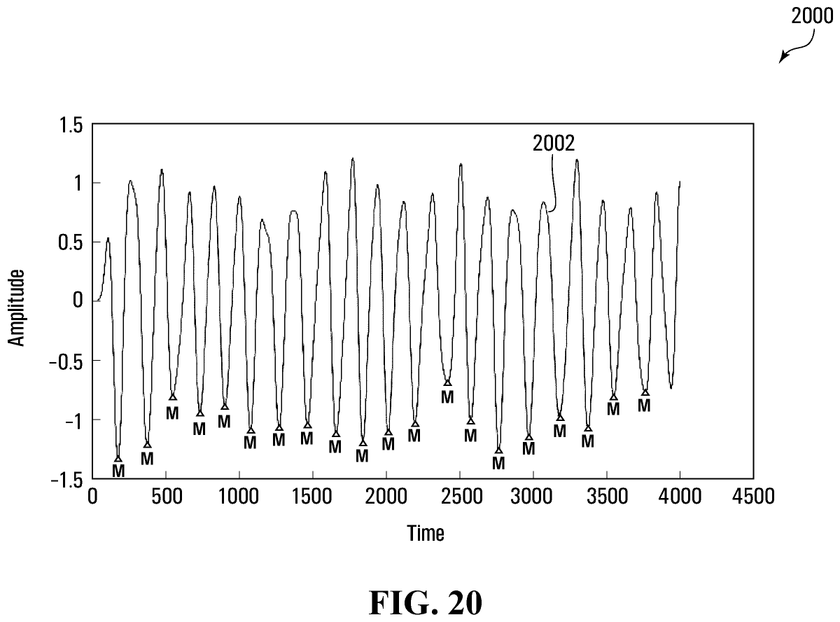

FIG. 19 is a trace of a rectified Signal for envelope detection, according to some embodiments. Note the preserved S1 and S2 heart sounds, indicated in FIG. 19 by characters X and O, respectively. A trace is indicated as 1902.

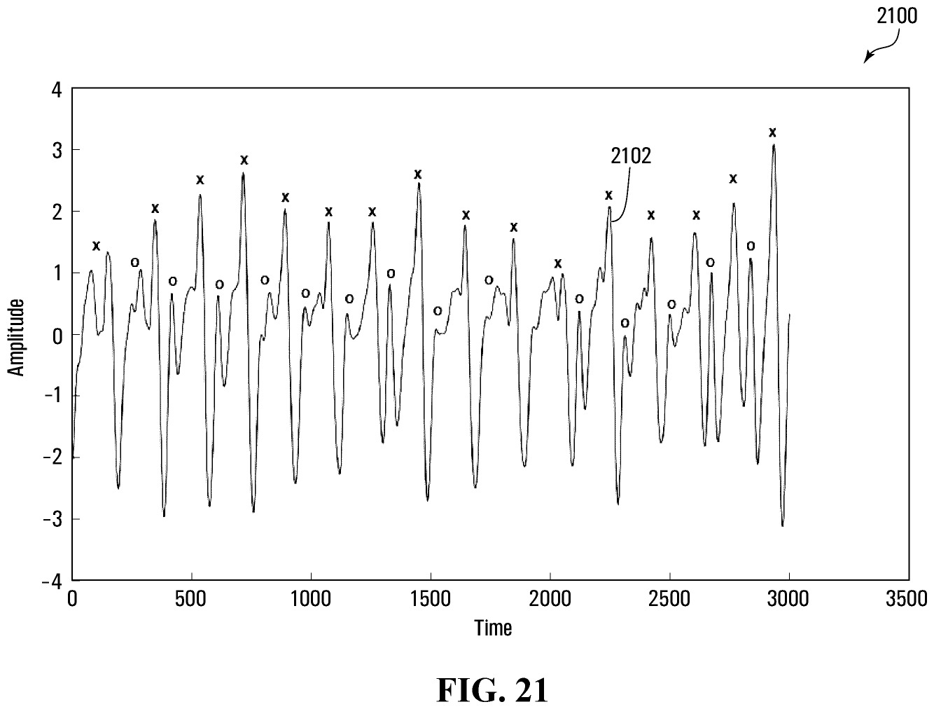

FIG. 20 is a trace of a low pass filtered signal at 2 Hz, and high passed signal at 0.7 Hz, according to some embodiments. Note how the S1 and S2 are made indistinct, and the minima are strongly distinguished in the signal allowing for beat-beat interval calculation. Minima are indicated in FIG. 20 as dots M at the minimum of each beat-beat cycle). A trace is indicated as 2002.

FIG. 21 is a trace of a filtered SCG signal, post rectification, low pass at 3 Hz, high pass at 0.7 Hz, according to some embodiments. Note that the signal preserves S1, and S2 peaks, indicated by characters X and O, respectively, in FIG. 21. A trace is indicated as 2102.

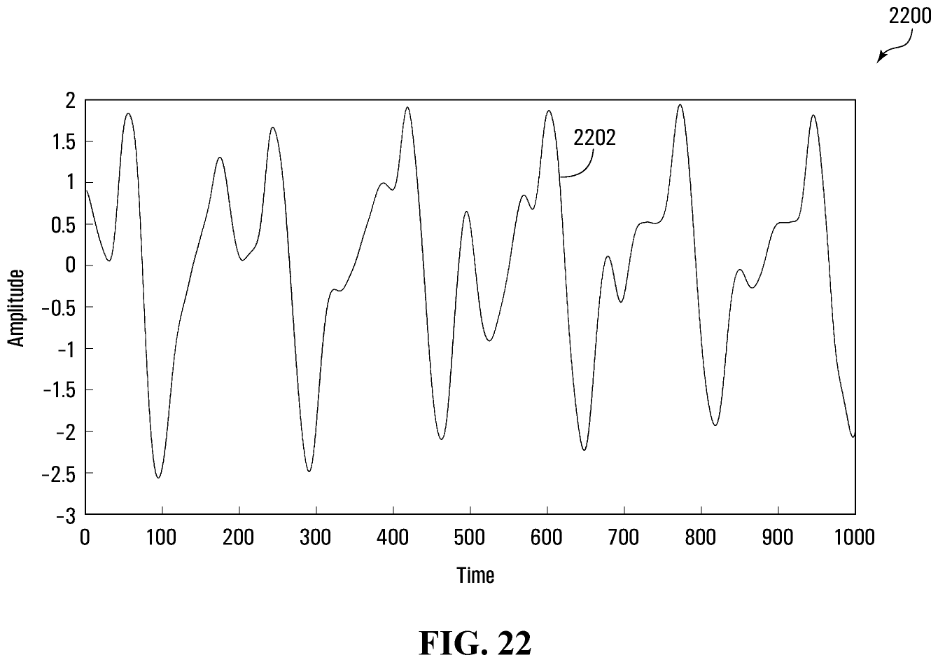

FIG. 22 is a trace of a 5 second segment of filtered data for template matching, according to some embodiments. Note the S1 as the high peaks, and S2 as the lower peak after the minima. A trace is indicated as 2202.

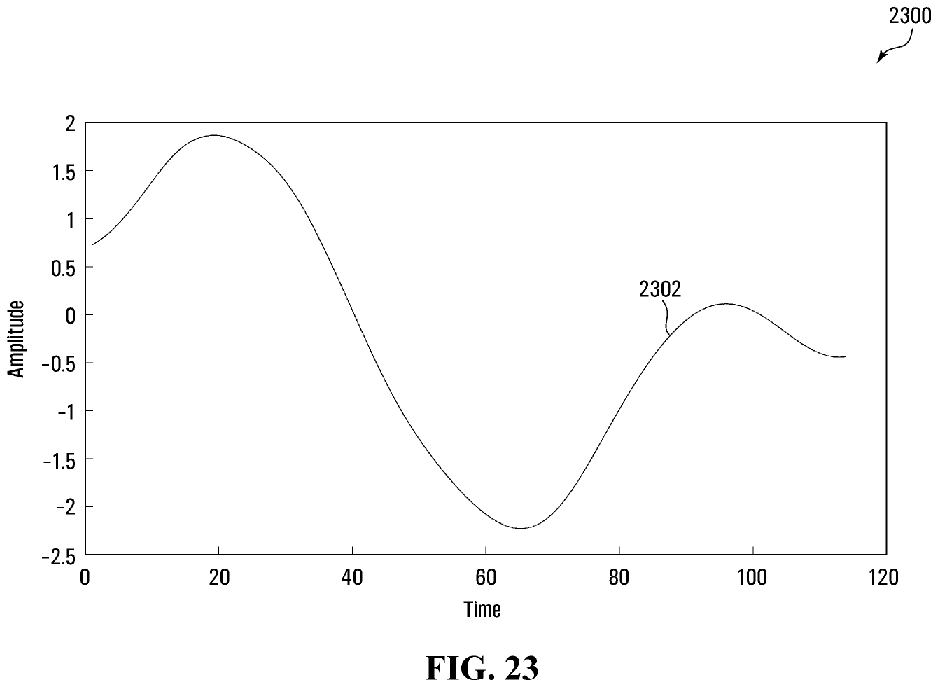

FIG. 23 is a trace of an extracted best match template from the above 5 second interval, according to some embodiments. A trace is indicated as 2302.

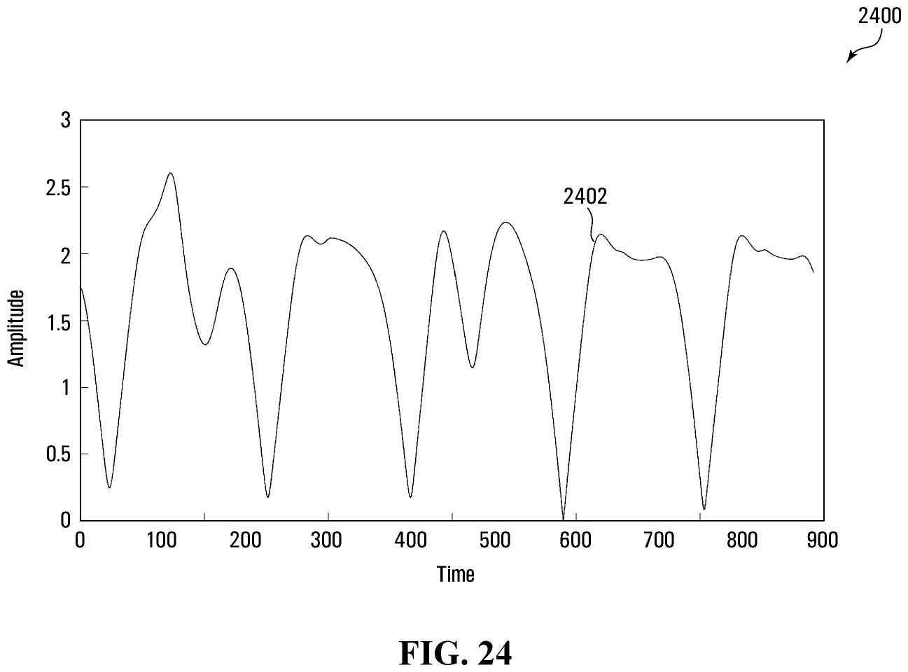

FIG. 24 is a trace of a correlation signal derived using cosine similarity between the template and the rectified five second signal window, according to some embodiments. As indicated in FIG. 24, the closer the correlation signal is to 0, the more similar the signal is to the template. The perfect match is the template (indicated at the 4th minima from the left). The correlation detection threshold filters out the smaller minima. The interval between the minima is the beat-beat interval that may be used for heart rate and heart rate variability derivation, according to some embodiments. A trace is indicated as 2402.

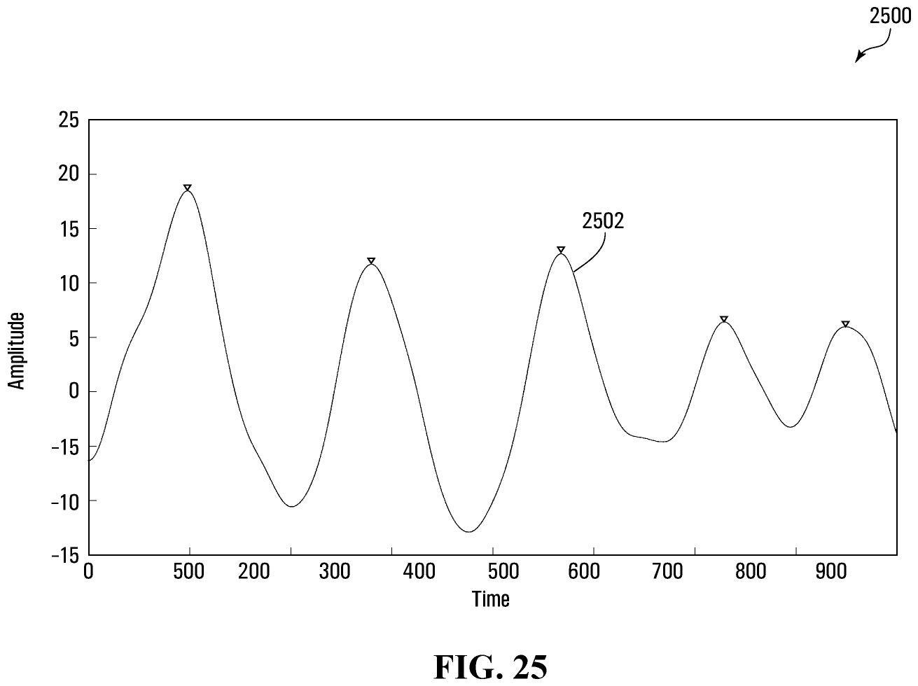

FIG. 25 is a trace of a derived respiration signal after filtering at 0.8 Hz low pass filter, and 0.1 Hz high pass filter, according to some embodiments. In this figure, pairs of Minima-Maxima are 1 breath cycle. A trace is indicated as 2502.

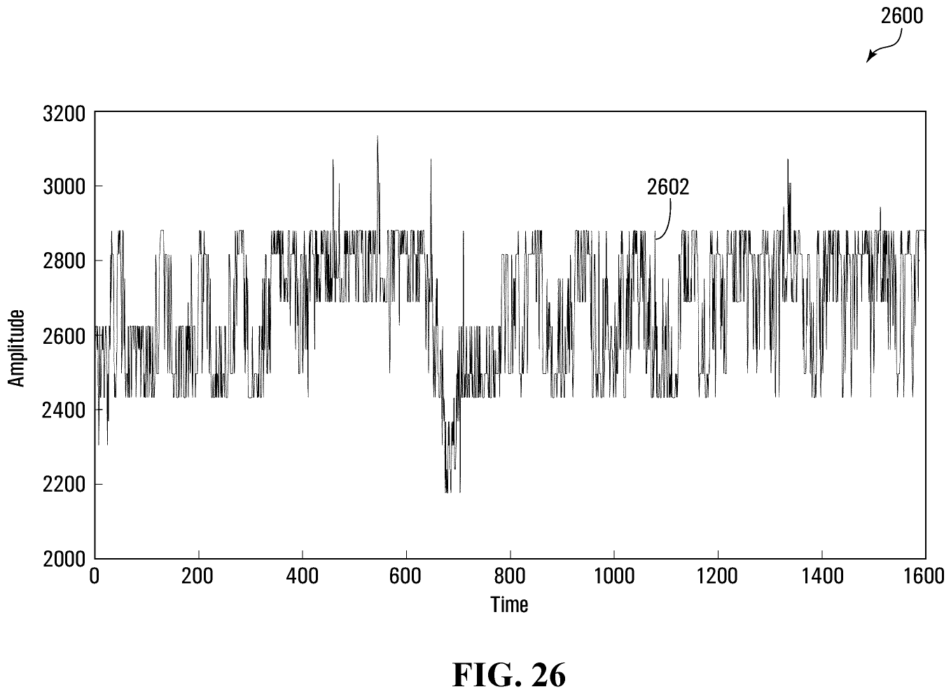

FIG. 26 is a trace of a raw Z axis signal at 25 Hz, with heavy aliasing noise. This trace is provided for comparison with FIG. 17, which was sampled at 800 Hz and has no aliasing colouring the respiration or the heart rate signals. A trace is indicated as 2602.

### Parameter Selection

As indicated in FIGS. 17-26, the sampling rate of the accelerometer may be varied, depending on a particular desired outcome. For example, sampling an accelerometer at a highest possible frequency may aid in preventing aliasing from obscuring low amplitude signals required for respiration and heart rate measurement.

The internal sample rate of the accelerometer may be higher than the sampling rate required for heart rate measurement (200 Hz). At 200 Hz, any frequencies above 100 Hz up to the Nyquist frequency of the native sample rate of the accelerometer will alias over the raw signal, which may affect the signal-to-noise ratio (SNR) of the signal.

Accordingly, in an effort to prevent and/or reduce aliasing, the signal may be oversampled at a higher frequency such as 800 Hz (800 Hz is selected in this example as the highest frequency for the accelerometer that was used for experimental purposes), and then decimated to 200 Hz.

As a result of oversampling and/or decimation, there may be an increase in the SNR of the heart rate and respiration signals, leading to potentially more accurate readings. A potential added benefit of decimating to a lower frequency (e.g., 200 Hz) for the heart rate derivations is that fewer calculations may be required, causing the process to be more efficient (e.g., in the 800 Hz to 200 Hz example, four times more efficient).

In some embodiments, acquiring the signal at the accelerometer's chip resolution may limit aliasing from higher frequencies, resulting in a signal with the highest signal to noise ratio (SNR). For low amplitude signals such as respiration rate and heart rate, this approach may facilitate obtaining a high level of accuracy for the sensing method.

For example, an approach may include, in accordance with some embodiments, decimating the signal to a frequency (e.g., 200 Hz) for heart rate calculation, to reduce the total number of calculations required by a substantial margin for template matching which at its best, is an O(N log(N)) algorithm and is generally a O(N²) algorithm.

In some embodiments, the signal may be decimated to a frequency (e.g., 25 Hz) for respiration and energy estimation, for example, to optimize for calculations as well by a factor (e.g., of 32) from the native frequency (e.g., 800 Hz), and to increase fixed point filter performance for filters that have a stop band cut off frequency at a ratio less than 1/1000 of the Nyquist frequency (e.g., 400 Hz), rendering them unstable at the native frequency. However, if using a processor with FPUs the filter stability is less of an issue.

For a beat detection algorithm, using minima, a signal envelope may be found through rectification and filtering as opposed to a Hilbert transform, for computational efficiency. A signal may be filtered (e.g., at 2 Hz) to blend the S1 and S2 heart sounds and to account for polymorphic behaviour, with regards to S1/S2 amplitude, murmurs, and time spacing. The filtering step may allow the minima between the end of a heart beat and the start of the next beat to become more pronounced, thereby allowing and/or facilitating determining and/or locating the heart rate, and the start location by finding the minima using a minima/peak detection algorithm.

The signal for the template matching may be filtered (e.g., at 3 Hz. Applicants explored various frequencies between 2-5 Hz, and found that 3 Hz was preferable) to preserve detail with regard to the S1 and S2 envelopes. This approach may aid in finding effective templates for beat detection by recognizing prominent S1/S2 pairs, in a noisy signal or when calculating heart rate variability metrics.

A 250 ms spacing may be chosen as the human heart rate is between 40 to 240 bpm in most human beings. Therefore a heartbeat is in most cases, at least 250 ms apart.

Further parameters were selected in the context of various cardiopulmonary characteristics.

For example, the heart rate was measured every 15 seconds to remove ectopic beats from the intervals collected in the past 15 seconds. The closer the time is to 60 seconds, the more accurate the beats per minute may be. Because the heart rate intervals may vary significantly at rest, 5 seconds may not be enough to get the most accurate heart rate estimate. However, at higher heart rates during activity or exercise, 5 seconds of data is more than enough, as the standard deviation between each heart rate interval may be at a minimum at faster beats per minute (BPMs).

Applicants selected 25 percent of the heart rate interval, experimentally. Applicants tested variations of 10 to 35 percent on gold standard ECG signals collected from multiple subjects; the number of real beats rejected was at a minimum, while the number of ectopic beats rejected was maximum. Applicants found that this approach yielded an efficient algorithm, as calculating 25 percent of the mean is a simple bit shift operation.

A 800 ms spacing may be chosen in relation to the respiration rate, as an at rest respiration rate may be between 12-20 bpm. A respiration rate below 12 or over 25 is considered abnormal. In general each breath is usually at least 800 ms leading to a max bpm of 75 bpm, which accounts for exercise, shortness of breath, or asthma. Since breathing can be consciously controlled, an individual could breathe faster than 500 ms, but the apparatus may not as concerned with picking up consciously fast breathing, as opposed to unconscious breathing.

Heart rate, heart rate variation and respiration rate can be derived from higher frequencies (e.g., frequencies above 200 Hz), and in some embodiments, 200 Hz is selected to minimize calculations on an embedded system, and to reduce battery consumption.

The application of a low-pass 2 Hz filter may be important as the low pass 2 Hz filter may be utilized to remove the polymorphic nature of the S1 and S2 sounds by blending the two together after finding the envelope of the raw SCG signal. The filtering causes the spaces between the S1 and S2 pair to become prominent, and easily detectable, allowing for more robust heart rate detection based on the minima-minima intervals.

Polymorphism of the SCG signal is a significant challenge. To account for polymorphism, the raw acceleration signal is filtered for a passband of 10 Hz-35 Hz to get the raw heart rate signals. The envelope of the signal is then determined, a low pass filter is applied the envelope at 2 Hz and the DC component is removed with a high pass filter at 0.7 Hz.

This approach aids in the removal of the polymorphic nature of S1 and S2 and blends the peaks somewhat together. The minima between two pairs of S1 and S2 (two heart beats) become highly prominent, allowing for more accurate, and efficient beat detection based on the minima that meet the adaptive threshold algorithm as described earlier.

Minima detection may be advantageous relative to peak detection as it may be less prone to noise, and the polymorphism of the S1 and S2 beats. Peak detection may become confused if filtered at 2 Hz, between the S1 and S2, causing the beat-to-beat intervals to vary more than the Minima-Minima intervals with reference to a gold standard ECG.

Once a cardiopulmonary signal is determined as described above, an output may be provided to a user. In some embodiments, such output may be a video, which may be pre-rendered and stored at computing device 110 and server 102.

For example, a library of pre-rendered videos may be stored at server 102 and may be accessible using calls to a function such as an application programming interface (API) at computing device 110.

Videos in the library may correspond to different heart rates. Typical heart rate levels used for the library may fall within the physiological range of human heart rates (e.g. 30 b.p.m. to 240 b.p.m.). In an example, pre-rendered videos may be available for heart rates of 45, 55, 65, 75, 85, 95, 105 and 115 b.p.m. In some embodiments, the heart rate intervals between pre-rendered videos may be constant. In other embodiments, the heart rate intervals may vary. For example, a library could contain pre-rendered videos for heart rate levels including 30, 60, 61, 62.5 and 200 b.p.m.

The number of pre-rendered videos stored in the library at server 102 and the range and values of heart rates to which the videos correspond may be selected to balance user experience with computational and storage requirements.

For each heart rate level in the pre-rendered video library, there can be one or more versions of videos at that heart rate level. For example, at a 60 bpm heart rate level, there may be a plurality of pre-rendered videos, each of the plurality of pre-rendered videos may possess different audio-visual effects/properties.

In the case of multiple pre-rendered videos being stored in a library for the same heart rate level, these files may, in one example, be selected at random and presented to the user after a heart rate measurement.

In another example, one of the multiple pre-rendered videos may be selected manually by the user if each file is linked/tagged to a specific effect. Effectively, different “filters” or settings can be selected by the user to return the desired video.

If filters or settings are in effect, the videos stored in the library for each heart rate level may be tagged with different types of filters or tags.

In some embodiments, for each heart rate level, the videos may possess unique or distinctive audio-visual effects or properties that are a function of the heart rate. For example, some videos can have audio-visual effects that are synchronized with the frequency of the recorded heart rate. For example, the speed and sound of a repeating animation can increase to match a higher heart rate. In another example, for elevated heart rate levels, the pre-rendered videos could have an increasingly red hue/filter, suggestive of an activated state, whereas relatively lower heart rate videos could have a blue hue, which is suggestive of a relatively calm state.

Other audio-visual effects may include sound type, sound intensity, speed of sound, colour intensities or any other audio-visual effect that is linked to a heart rate level or other biosignal. The audio-visual effects to be used in each video may be determined by the developer/designer of the system to achieve the desired effect.

Other metadata can be linked to or associated with each video in the library. In addition to filter types or tags, text may be associated with the video. Further, if sent to another user using the application, the frequency of the heart rate could be transmitted to a receiving application, and other biofeedback modalities such as haptic feedback may be used by passing the heart rate frequency of the shared video to a haptic motor to pulse/vibrate at the desired frequency.

Pre-rendered videos may be stored locally on a computing device 110 and such local copies may be periodically updated to match a video library at server 102.

For example, the first time a user signs into an application at computing device 110, there may be no pre-rendered videos associated with the application at the computing device 110. In other words, videos may not initially be stored locally at computing device 110 and may not be integrated with the application. As part of the initialization of the application, a library of pre-rendered videos may be partially or fully downloaded from server 102.

After the user records a heart rate via phone-based mechanical heart rate acquisition (e.g. ballistocardiography, seismocardiography), a pre-rendered video linked to the heart rate level closest to measured heart rate is accessed and played to the user so the user can preview the pre-rendered video.

The user may have the option to share the pre-rendered video via email, SMS, acceptable social media platforms (e.g. Instagram, Facebook), or other applications that support file sharing (e.g. Slack, Trello, Notes, WhatsApp, or the like).

Each time the user starts a new session within the application, a check is made (e.g. with an API call) to see if any updates/modifications have been made to the pre-rendered video library at server 102. If no updates are available, no videos are downloaded to the device. If modifications have been made or differences between the server side library and the local pre-rendered video library otherwise exists, these changes are effected in the local pre-rendered video library such that the local pre-rendered video library reflects the changes made to the rendered server video library. If new pre-rendered videos are present in the library (e.g. for new heart rates), those videos may be downloaded to computing device 110, either immediately, at a scheduled time, or when a measured heart rate falls close to the new pre-rendered heart rate.

Storing pre-rendered videos on server 102 may provide flexibility, as videos can be easily added or removed and such changes may be reflected at computing device 110 through a function such as an API call. Caching the pre-rendered videos at computing device 110 may provide a reduction in data usage, since only new videos are downloaded whenever there is an update on the server side. Additionally, the size of the application may be smaller and can be downloaded and installed onto the user's mobile device in less time.

Storage of pre-rendered videos on the server may also allow for videos to be presented to the user in less time, as rendering the video (audio and visual components) has already completed. The playing of pre-rendered videos is less computationally intensive and allows for higher video quality, as compared to rendering on-demand. In addition, audio effects can be pre-made to match a desired effect. For example, generating an audio profile to make a custom animation that synchronizes with a specific heart rate on the fly is challenging, as the frequency of the heart rate will alter the base audio file pitch, which may result in undesirable distortions in the sound. Corrections to the audio pitch can be made, but these corrections may require computationally expensive calculations or processing.

Furthermore, pre-rendered videos stored at server 102 may be shared with other computing devices 110 by passing resource locators pointing to server 102. For example, a JSON object-containing library indexing information of the pre-rendered video could be passed to the receiving application, instead of transferring the media data itself. This allows for the recipient and the sender to use less data, and videos can be shared faster between application users with less data usage between the sender and recipient. This in turn may allow for richer video/media content to be used.

As noted, data from data acquisition device 114 may be provided to server 102 for storage in a data structure within storage 104. Data may be provided from data acquisition device 114 to server 102 by way of network 106. In some embodiments, data may be transmitted first from data acquisition device 114 to an associated client computing device 110 and subsequently from the client computing device 110. Alternatively, data acquisition device 114 may transmit data directly to server 102.

Data transmitted to server 102 may include, for example, respiration rate, respiration rate variability, heart rate, heart rate variability, and a user identifier. Data may be sent as a series of measurements, each of which may also include an associated time stamp identifying the time at which the measurement was taken.

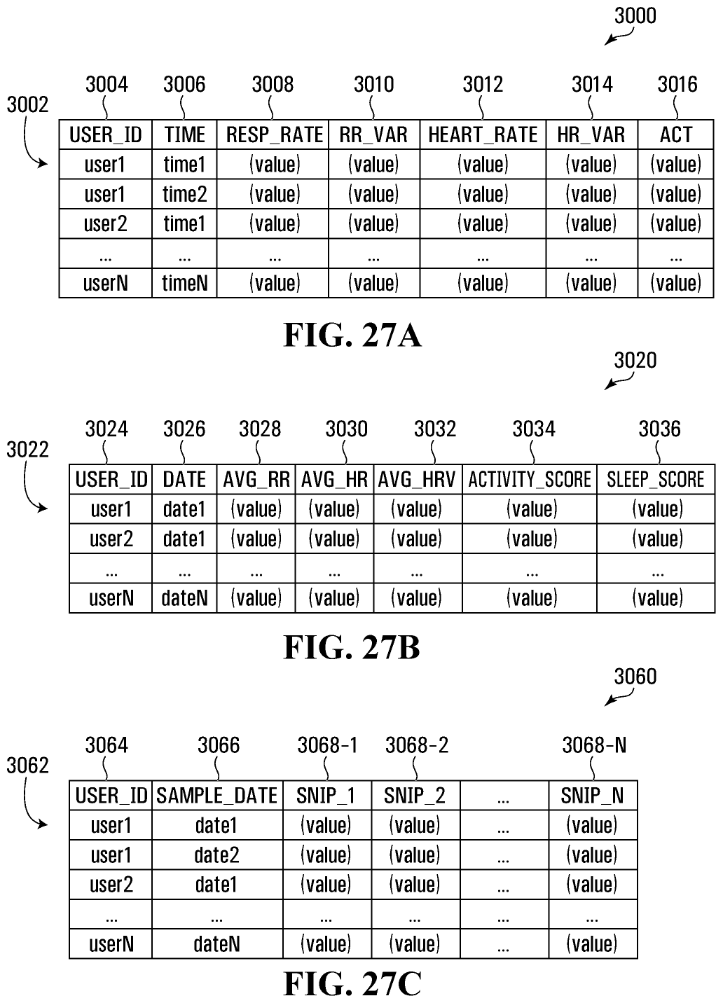

FIG. 27A depicts an example table 3000 within storage 104 containing data from a plurality of data acquisition devices 114. Table 3000 includes a plurality of rows 3002, each of which corresponds to a set of measurements received from a data acquisition device 114. Each row contains entries in a user id field 3004 and a time field 3006, which identify the user 116 with whom the measurements are associated and the time at which the measurement was taken; a respiration rate field 3008; a respiration rate variability field 3010; a heart rate field 3012, a heart rate variability field 3014 and an activity classification field 3016, each of which contains a measurement or average value obtained from data acquisition device during a time period corresponding to the value in time field 3006.

User identifiers in field 3002 may be included in messages transmitted to server 102, or may be populated by server 102 based on data stored at the server. For example, a user 116 may log in to a mobile computing device 110, which may transmit measured data along with credentials. Alternatively, each data acquisition device 114 may be associated with a user, and a corresponding user ID may be added to each message received from the data acquisition device 114. As depicted, data for multiple users 1, 2 . . . N are stored in a single table. Alternatively, user data may be segmented into multiple tables, for example, one table per user.

Table 3000 may therefore contain a set of historical measurements of user data for one or more users 116.

Server 102 may also store one or more tables containing values derived from measurements stored in table 3000.

For example, FIG. 27B depicts a table 3020 containing a series of daily scores for users 116. Specifically, table 3020 includes a plurality of rows 3022, each of which contains values in a user ID field 3024, a date field 3026, average respiration rate and heart rate fields 3028, 3030, an average heart rate variability field 3032, and activity and sleep score fields 3034, 3036.

Each row 3022 of table 3020 corresponds to a specific user 116 and date, and each of of average respiration rate and heart rate fields 3028, 3030, average heart rate variability field 3032, and activity and sleep score fields 3034, 3036 contains a value calculated from measurements in table 3000 for the corresponding user and day.

Average respiration rate and heart rate fields 3028, 3030 may contain an arithmetic average of respiration rate and heart rate measurements taken by data acquisition device and recorded in table 3000 for a particular day. Heart rate variability field 3032 may contain a measurement of the variability of heart rate measurements for the particular day.

Activity score 3034 is an index of the activity level of user 116 throughout the day. In an example, activity score 3034 may represent a total duration of time during which user 116 was determined to be moving. Optionally, activity score 3034 may be a weighted score, with periods of low-intensity movement assigned a first weight, and periods of high-intensity movement assigned a second, higher weight. Accordingly, the value of activity score 3034 is representative of a quantity of physical exercise by a user 116 during a particular day.

Sleep score 3036 is an index of the quantity or quality of sleep by a user 116 on a particular day. In an example, sleep score 3036 may be based on the total duration of time during which a user 116 is determined to be not moving, and the respiration rate and heart rate of the user 116 are below threshold values. Optionally, sleep score 3036 may also take into account the variability of heart rate and respiration rate during such periods. Accordingly, sleep score 3036 may represent both quantity of sleep and quality of sleep (e.g. sleep stage).

Sleep score 3036 may be derived based on metrics calculated from measurements obtained by data acquisition device 114. Such calculations may be performed at data acquisition device 114, mobile computing device 110, server 102, or a combination thereof.

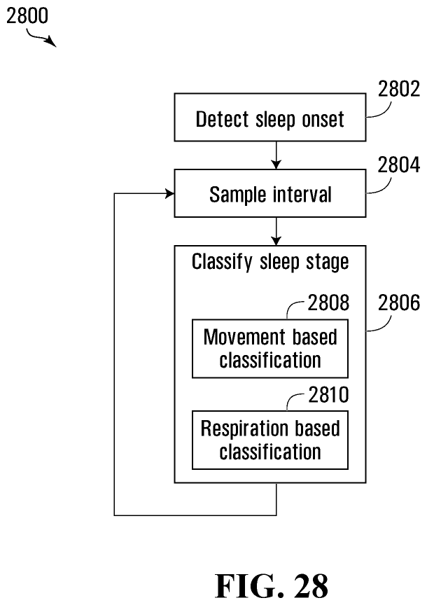

FIG. 28 is a flow chart depicting a process 2800 of sleep monitoring to determine sleep score 3036. At block 2802, measurements from data acquisition device 114 is monitored to identify the onset of sleep. Sleep onset may be identified based on the user's total movement energy and standard deviation of total movement energy. Total movement energy may be measured as the root mean of the sum of the squared values in the filtered signals from the X, Y and Z-axis acceleration measurements obtained by data acquisition device 114. Onset of sleep is identified when the standard deviation reaches a minimum and the slope of the movement energy has fallen below a determined threshold.

At block 2804, measurements from data acquisition device 114 are monitored for a sampling period. In some embodiments, the sampling period is approximately four minutes.

At block 2806, measurements from the sampling period are used to classify a sleep stage during that period. Classification may include movement-based classification at block 2808 and metric based classification at block 2810.

At block 2808, an actigraphy value is determined for the sampling period. The actigraphy value may be, for example, a mean peak-to-trough value of an acceleration signal on one of the X, Y and Z axes. In some examples, the axis selected for the actigraphy value is the axis with the largest peak-to-trough mean. The actigraphy value is compared to the actigraphy value for the preceding interval. If the difference is above a threshold, the interval is classified as a wake interval. If the difference is close to zero, the interval is classified as a rapid-eye-movement (REM) interval.

At block 2810, respiration and time-based metrics are computed for the interval. The metrics may include all or a subset of: mean and standard deviation breath interval time, interquartile breath time, greatest breath time, mean and standard deviation of breath amplitude differences, interquartile range of breath amplitude differences, smallest peak to trough difference, greatest power from PSD, sum of the 2 frequency bins containing the greatest power, sum of the total power+−0.05 Hz of the user average breathing frequency (˜14 breaths per minute), sum of the total power in bins −0.1 to −0.05 Hz of the user average breathing frequency and in bins +0.05 to +0.1 Hz of the user average breathing frequency, sum of the total power above average breathing frequency +0.2 Hz, root mean squared successive differences (RMSSD) of breath time intervals, RMSSD of breath amplitude differences, mean and standard deviation of inhale amplitudes, mean and standard deviation of exhale amplitudes, RMSSD of inhale amplitude differences, RMSSD of exhale amplitude differences, previous interval sleep stage, and indicators if the previous interval was either an apnea or hypopnea interval, whether the interval is a REM interval.

The metrics are then provided as inputs to a machine learning model to determine a sleep classification. The machine learning model may be a random forest model. In some examples, the random forest model may be based on 500 trees, which may be trained based on a sleep databases such as the publicly available Physionet database. Alternatively, other types of models could be used, such as an artificial neural network.

Sleep score 3036 may be based on based on cumulative time in each sleep stage, the number of wakenings, sleep onset time, and the like.

Optionally, sleep classification may further be based on metrics of heart rate variability, such as standard deviation of the NN intervals (SDNN), root mean squared successive differences (RMSSD), number of NN intervals longer than 50 ms (NN50), percentage of intervals longer than 50 ms (pNN50), median RR interval size, interquartile range, average deviation of the RR intervals from the mean RR interval time, mean and standard deviation of heart rates evaluated from the berger algorithm, maximum and minimum berger heart rates, sum of absolute deviations of successive berger heart rates, number of successive berger heart rate deviations greater than 5 beats per minute, number of successive berger heart rate deviations greater than 10 beats per minute, and the power law constant and the r squared parameter evaluated from detrended fluctuation analysis.

Optionally, sleep classification may further be based on spectral domain metrics such as: sum of power in very low frequency range (<=0.04 Hz), sum of power in low frequency range (0.04-0.08 Hz), sum of power in medium frequency range (0.08-0.15 Hz), sum of power in high frequency range (0.15-0.4 Hz) (as well as the normalized measures for these frequency ranges), low frequency to high frequency ratio, sum of the power from the 2 largest low frequency bins, sum of the power from the 2 largest high frequency bin, frequency of the bin with the greatest power, greatest power of all of the bins.

In some embodiments, additional scores may be derived from data obtained by data acquisition device 114. For example, scores representative of cardiovascular health may be derived from heart rate, heart rate variability, body mass index and activity data for a user. Scores representative of psychophysiological stress may be derived from respiratory and cardiac patterns throughout the day, such as during the night and just before waking. Scores representative of mindfulness may be derived from cardiac and respiratory patterns used to classify meditative, calm or focused states and recording duration of such states throughout the day.

Server 102 may also receive genetic information from genetic data acquisition facility. Genetic information may be stored at server 102 in one or more tables. FIG. 27C depicts an example table 3060.

Table 3060 has a plurality of rows 3062, each of which contains values in a user ID field 3064, a sample date field 3066, and n genetic SNP fields 3068-1 through 3068-n (individually and collectively, SNP fields 3068). Each row 3062 contains information associated with a genetic sample from a user 116 on a specific date. For some users 116, table 3060 may include multiple rows 3062, corresponding to samples on different dates. For example, FIG. 27C depicts two rows containing data from samples of user1 taken on two different dates, date1 and date2.

Thus, using values stored in table 3060, analytics engine 132 of server 102 can observe and quantify changes in genetic information of users 116. For example, if telomere length is among the genetic data recorded in table 3060, server 102 can determine changes in telomere length for a user over time. Studies have shown that telomere length, specifically, decreased telomere length, is associated with aging, and more particularly, with oxidative stress. Accordingly, using table 3060, server 102 can construct integrated models of cumulative oxidative stress in tandem with data collected by data acquisition device 114 to model aging of a user 116.

Similarly, in some embodiments, analytics engine 132 may observe and quantify changes in methylation pattern, histone modification, and microRNA. These changes influence which genes are expressed, and how highly a given gene is expressed. Many behavioral and environmental influences, such as diet, exercise, and sun exposure, can contribute to these kinds of epigenetic modifications, which are themselves often associated with disease risk and incidence. Monitoring these epigenetic changes in the population as a function of biosensor or smart phone derived traits (location/pollution levels) can indicate which behaviors are best at inducing the preferred epigenetic state.

Thus, in tables 3000, 3020, 3060, server 102 maintains a repository of dynamic physiological and behavioural measurements obtained by the accelerometer-based data acquisition device 114 and data derived from such measurements, as well as genetic data obtained from data acquisition facility 112. Data in tables 3000, 3020, 3060 is user specific.

Analytics engine 128 of server 102 is further configured to obtain population-level data by analysis of groups of users 116. Users 116 may be grouped, for example, according to shared gene mutuations reflected in SNP fields 3068, shared behavioural or physiological characteristics, such as sleep amount or quality, resting heart rate, demographics (i.e. age, gender, location) etc. Some groups may be defined using ranges. That is, individuals with average heart rate above a first threshold and below a second threshold may be placed in a common group.

Analytics engine 128 is further configured to analyze user-specific and population-level data to identify correlations and to provide feedback to users 116. For example, data may be analyzed to define groups based on data obtained from data acquisition device 114, genetic data, or a combination thereof. The resulting group may be analyzed to determine commonalities among individuals of the group. For example, data from data acquisition device 114 may be used to identify a group of individuals with high sleep latency. Genetic data may be analyzed for members of that group to identify correlations of genetic data with long sleep latency. In another example, genetic data may be used to identify a group with abnormally short telomere length. Data from data acquisition device 114 may then be analyzed to identify, e.g. behavioural or physiological characteristics correlated with short telomere length. Such analysis may be conducted using suitable machine-learning algorithms, such as supervised or unsupervised machine learning, and statistical analysis tools, such as regressions and the like. Correlations identified may be stored in association records at server 102. Correlations may be used to identify and provide relevant feedback to individuals, such as recommended activity changes or the like.

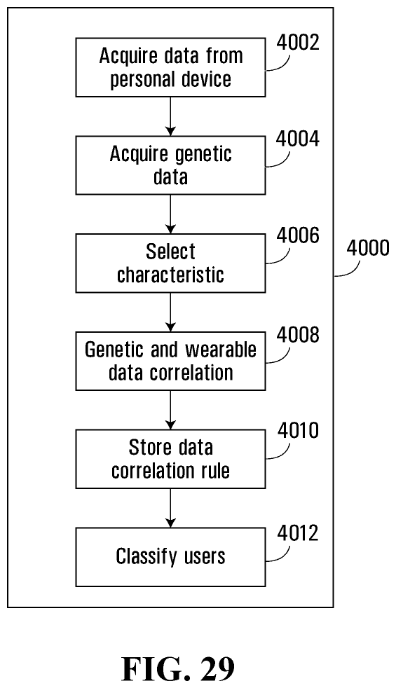

FIG. 29 depicts an example process 4000 of creating a data association record. At block 4002, physiological data and behavioural data is acquired from a data acquisition device 114 associated with a user 116. The acquired data is stored in table 3000 at server 102. Analytics engine 128 derives additional data from the measured values, such as sleep quality and exercise data, and stores the derived data in table 3020.

At block 4004, genetic data is acquired from a genetic data acquisition facility 112 and sent to server 102 by way of network 106, where it is received and stored in table 3020.

At block 4006, a characteristic is selected for which it is desired to provide feedback to users 116. For example, sleep score may be selected if it is desired to provide feedback to users on how to improve sleep scores.

At block 4008, analytics engine 128 performs an analysis of data in tables 3000, 3020, 3060 to identify correlations between data in the tables and the selected characteristic. As noted, the analysis may include various techniques such as supervised or unsupervised machine learning, regression analysis, and the like.

The analysis performed at block 4008 may reveal statistical correlations between values in fields of tables 3000, 3020, 3060 and the characteristic selected at block 4006. For example, the analysis performed at block 4008 may reveal that a particular genetic mutation correlates to poor sleep quality, particularly among users with activity level scores below a certain threshold.

At block 4010, discovered correlations are translated into association records reflecting relationships between variables. Association records may be logic statements prescribing feedback to be sent to users meeting a certain set of criteria. For example, if a particular genetic mutation and exercise below a certain threshold are found to correlate to poor sleep quality, all users having that genetic mutation and exercise scores below that threshold are assigned to a group so that feedback can be provided collectively to members of that group.

Alternatively, data correlation rules may be stored as encoded representations of sets of variables and strength with which they are correlated. For instance, in the above-described example, a data correlation rule may specify that a genetic SNP field 3068 having a particular value and activity score field 3034 having a value above a threshold are correlated with sleep score field 3036, with a particular confidence or strength of the correlation.

At block 4012, users 116 may be classified based on correlation rules. For example, users may be placed into a group considered to be at elevated risk for poor sleep quality by virtue of possessing a genetic mutation and having activity levels below a threshold. Groups may be defined by creation of a “group” field in one or more of tables 3000, 3020, 3060, identifying groups to which a user 116 belongs. Alternatively, groups may be defined as requests, e.g. SQL join requests.

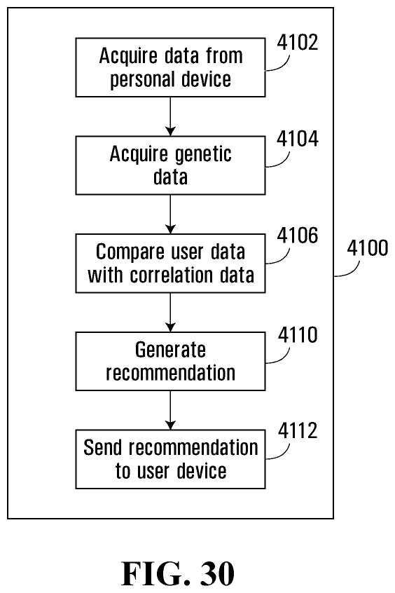

Using the correlation rules created and stored at block 4010, analytics engine 128 may provide feedback to individual users 116. FIG. 30 depicts an example process 4100 of providing feedback.

At block 4102, physiological and behavioural data is acquired from a data acquisition device 114 associated with a user 116. The acquired data is stored in table 3000 at server 102. Analytics engine 128 derives additional data from the measured values, such as sleep quality and exercise data, and stores the derived data in table 3020.

At block 4104, genetic data is acquired from a genetic data acquisition facility 112 and sent to server 102 by way of network 106, where it is received and stored in table 3020.

At block 4106, data acquired and derived for user 116 is compared with correlation rules stored at server 102 to determine if the user belongs to any groups for which correlation rules have been stored. For example, entries in table 3020 are used to compare nucleotide mutations present in user 116 to correlation rules stored at server 102. Thus, the analysis performed at block 4106 determines if user 116 belongs to a population which has been correlated with a characteristic of interest, such as sleep quality, aging or the like. Physiological and behavioural data of user 116 is compared to the correlation rule. That is, if an correlation rule specifies that a particular genetic mutation is associated with poor sleep quality if activity score is below a threshold, user 116's data is compared to the threshold.

At block 4110, feedback is created in the form of a recommendation for user 116, based on the comparison of user 116's data to the correlation rule. For example, if the user's activity score is below the threshold defined by the correlation rule, analytics engine 128 may create a recommendation saying “get more exercise to improve sleep”. The recommendation may be automatically created using natural language algorithms. Recommendations delivered in natural language may be viewed more favourably by users, as opposed to messages that appear to be computer-generated.

Comparison of user 116's data to stored correlation rules and generation of a recommendation may be include assigning user 116 to one of a plurality of “bins” each corresponding to a range of the variable being examined. For example, a population having a particular genetic mutation may be sorted into bins corresponding to ranges of activity scores, with each bin being associated with a different probability of sleep quality. Recommendations may be pre-generated for each bin.

At block 4112, the generated recommendation is sent to user 116. In some embodiments, the message is delivered to a mobile computing device 110 associated with the user. The message may be delivered over network 112 to an application at device 110, using short-messaging service (SMS), or any other suitable method. In some embodiments, the message may delivered in text form. Alternatively, the message may be delivered in audio form, for example, using a text-to-speech engine.

In some embodiments, part or all of processes 4000, 4100 may be performed at data acquisition device 114 or at mobile computing device 110, rather than server 102.

As depicted, server 102 collects physiological and behavioural data from a data acquisition device 114 coupled to (e.g. worn by) the user 116. Measurements are taken by operation of accelerometers providing acceleration signals in three different directions. Measurements may be transmitted to mobile computing device 110 of user and subsequently to server 102. In other embodiments, physiological and behaviour may be directly measured by mobile computing device 110. For example, mobile computing devices such as smartphones often have physical sensors including accelerometers. Accelerometer traces from a smartphone may be used substantially as described above to obtain measurements of respiration and heart rate and variability, from which more complex data may be derived, such as sleep quality and activity level.

In some embodiments, server 102 may also store one or more tables with additional data associated with users 116. For example, server 102 may store positioning data obtained from a GPS trace or the like, which may be part of data acquisition device 114 or mobile computing device 110. Server 102 may also store nutritional data, which may for example be input by users 116 using mobile computing devices 110.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the detailed embodiments described above and illustrated are examples only. The invention is defined by the appended claims.

What is claimed is:

1. An apparatus for measuring cardiopulmonary data of a wearer, comprising:
a sensor operable to produce a data stream indicative of movements of a wearer's body;
a positioning device holding said sensor proximate an anatomical landmark on said wearer's body for conduction of mechanical vibrations from said wearer's body to said sensor; and
a processor configured to receive said data stream and produce a rate signal indicative of cardiac or respiratory rate data of said wearer using an algorithm comprising peak detection.

2. The apparatus of claim 1, wherein said positioning device includes at least one of a pocket formed in a garment, a magnetic attachment, and a clip.

3. The apparatus of claim 2, wherein said garment is a shirt.

4. The apparatus of claim 1, wherein said pattern detection algorithm comprises template matching.

5. The apparatus of claim 1, wherein said data stream comprises measurements sampled at a first frequency and said processor is configured to decimate said signal to a second frequency lower than said first frequency.

6. The apparatus of claim 1, wherein said processor is configured to produce said rate signal according to one of a first data processing mode and a second data processing mode less computationally intensive than said first data processing mode, and to select between said first and second processing modes by processing said data stream according to a heuristic relating said data stream to user activity.

7. The apparatus of claim 1, wherein said rate signal is representative of one or more of respiration rate, heart rate and heart rate variability.

8. The apparatus of claim 1, wherein said rate signal is representative of all of respiration rate, heart rate and heart rate variability.

9. The apparatus of claim 1, wherein said sensor is a MEMS accelerometer.

10. The apparatus of claim 1, wherein said sensor is biased against said wearer's chest by gravity.

11. The apparatus of claim 1, further comprising a wireless radio for transmitting said rate signal to a computing device.

12. An apparatus for measuring cardiopulmonary data of a wearer, comprising:
a sensor mounted against a wearer's body to produce a data stream indicative of movements of said wearer's body;
a processor configured to receive said data stream and produce a rate signal indicative of cardiac or respiratory rate data of said wearer by correlating segments of said data stream to templates using an algorithm comprising peak detection;
a wireless radio for transmitting said rate signal to a computing device.

13. The apparatus of claim 12, wherein said sensor is operable to produce said data stream by sampling at a first frequency and said processor is configured to decimate said signal to a second frequency lower than said first frequency.

14. The apparatus of claim 12, wherein said processor is configured to produce said rate signal according to one of a first data processing mode and a second data processing mode less computationally intensive than said first data processing mode, and to select between said first and second processing modes by processing said data stream according to a heuristic relating said data stream to user activity

15. The apparatus of claim 14, wherein said heuristic comprises decimation of said data stream and calculating an estimated activity level from cumulative acceleration measurements taken by said sensor.

16. The apparatus of claim 12, wherein said rate signal is representative of one or more of respiration rate, heart rate and heart rate variability.

17. The apparatus of claim 12, wherein said rate signal is representative of all of respiration rate, heart rate and heart rate variability

18. The apparatus of claim 12, wherein said processor is configured to produce said rate signal without performing floating point operations, by converting floating point numbers to a fixed-point number format.

19. An apparatus for measuring cardiopulmonary data of a wearer, comprising:
a sensor for producing a data stream indicative of cardiac, activity classification, activity level or respiratory data;
a processor configured to receive said data stream and produce a rate signal indicative of cardiac or respiratory rate data of said wearer according to one of a first algorithm and a second algorithm less computationally intensive than said first algorithm, wherein said processor is configured to select one of said first and said second algorithms by processing said data stream according to a heuristic relating said data stream to an activity level of a wearer.

20. The apparatus of claim 19, wherein said heuristic comprises estimating integrals derived from measurements from said sensor indicative of activity of said wearer.

21. The apparatus of claim 20, wherein said heuristic comprises decimation of said data stream.

22. The apparatus of claim 21, wherein said heuristic comprises estimating integrals derived from acceleration measurements.

23. A method of providing health information to a user, comprising:
receiving a first data set comprising measurements of bodily movements obtained from an accelerometer mounted to the user's body;
receiving a second data set comprising genetic data associated with said user;
storing said first and second data sets in respective first and second tables in a data store;
performing a correlation analysis to identify an association between data of said first table and data of said second table;
storing, in said data store, a rule representative of said association;
generating a recommendation by comparing said data of said first and second data set to said rule, and transmitting said recommendation to a mobile computing device of said user by way of a communication network.

24. The method of claim 23, wherein said first and second tables contain data related to a plurality of users, and wherein said performing a correlation analysis comprises correlating characteristics of said users in said first table to characteristics of said users in said second table.

25. The method of claim 24, wherein said performing a correlation analysis comprises a machine learning algorithm.

26. The method of claim 23, further comprising deriving at least one of physiological data and behavioural data from said first data set and storing the derived data in said data store.

27. The method of claim 23, wherein said genetic data comprises telomere length.

28. The method of claim 23 wherein said comparing comprises assigning said user to a bin based on values in said first data set.

29. The method of claim 23, wherein said comparing comprises assigning said user to a group based on values in said second data set and comparing said values of said first data set to a rule applicable to said group.

30. The method of claim 23, wherein said first data set is received from a smart phone and said recommendation is transmitted to a smart phone.

31. The method of claim 23, comprising deriving a sleep score value based on values of said first data table and performing a correlation analysis to identify an association between said sleep score value and data of said second table.

32. The method of claim 31, wherein said deriving a sleep score value comprises identifying a sleep onset based on a calculation of movement energy.

33. The method of claim 31, wherein said deriving a sleep score comprises classifying a sleep stage based on a metric of respiration.

34. A system for acquisition and analysis of health data, comprising:
a data acquisition device comprising an accelerometer for measuring movements of a user's body, and operable to electronically transmit a data set representing said movements;
a data store with a first table for containing said data set and a second table containing genetic data of the user;
a processor;
a memory containing computer-readable instructions which, when exercised by said processor, cause said processor to:
receive said data set by way of said network;
perform a correlation analysis to identify an association between data of said first table and data of said second table;
store, in said data store, a rule representative of said association;
generate a recommendation by comparing said data of said data set and said genetic data of said user to said rule, and transmit said recommendation to a mobile computing device of said user by way of a communication network.

35. The system of claim 34, wherein said first and second tables contain data related to a plurality of users, and wherein said computer-readable instructions cause said processor to perform a correlation analysis by correlating characteristics of said users in said first table to characteristics of said population in said second table.

36. The system of claim 34, wherein said instructions cause said processor to perform a correlation analysis comprises a using a machine learning algorithm.

37. The system of claim 34, wherein said instructions cause said processor to derive at least one of physiological data and behavioural data from said data set and store the derived data in said data store.

38. The system of claim 34, wherein said genetic data comprises telomere length.

39. The system of claim 34 wherein instructions cause said processor to assign said user to a bin based on values in said data set.

40. The system of claim 34, wherein said instructions cause said processor to assign said user to a group based on said genetic data and to compare said values of said data set to a rule applicable to said group.

41. The system of claim 34, wherein said data set is received from a smart phone and said recommendation is transmitted to a smart phone.

42. The system of claim 34, wherein said computer-readable instructions cause said processor to derive a sleep score value based on values of said data set and perform a correlation analysis to identify an association between said sleep score value and data of said second table.

43. The system of claim 42, wherein said computer-readable instructions cause said processor to derive a sleep score value comprises identifying a sleep onset based on a calculation of movement energy.

44. The system of claim 43, wherein said computer-readable instructions cause said processor to derive a sleep score comprises classifying a sleep stage based on a metric of respiration.

45. An apparatus for measuring cardiopulmonary data of a wearer, comprising:
a sensor operable to produce a data stream indicative of movements of a wearer's body while positioned proximate an anatomical landmark on said wearer's body for conduction of mechanical vibrations from said wearer's body to said sensor;
a processor configured to receive said data stream and produce a rate signal indicative of cardiac or respiratory rate data of said wearer using an algorithm comprising peak detection;
a display for presenting feedback based on said data stream.

46. A method of measuring cardiopulmonary data of a wearer, comprising:
positioning a data acquisition device comprising a sensor proximate an anatomical landmark on said wearer's body for conduction of mechanical vibrations from said wearer's body to said sensor; to produce a data stream indicative of movements of said wearer's body;
at a processor, receiving said data stream and producing a rate signal indicative of cardiac or respiratory rate data of said wearer using an algorithm comprising peak detection; and
presenting feedback based on said data stream on a display of said data acquisition device.

47. The method of claim 46, further comprising a positioning device holding said sensor proximate an anatomical landmark on said wearer's body for conduction of mechanical vibrations from said wearer's body to said sensor.
